(19) United States
(12) Patent Application Publication
(10) Pub. No.: US 2020/0239559 A1
(43) Pub. Date: Jul. 30, 2020

(54) ANTI IGF, ANTI PD-1, ANTI-CANCER COMBINATION THERAPY

(71) Applicant: Boehringer Ingelheim International GmbH, Ingelheim am Rhein (DE)

(72) Inventors: Ulrike WEYER-CZERNILOFSKY, Baden (AT); Markus RESCHKE, Birsfelden (CH)

(21) Appl. No.: 16/649,763

(22) PCT Filed: Sep. 28, 2018
(86) PCT No.: PCT/EP2018/076494

§ 371 Date: Mar. 23, 2020

(30) Foreign Application Priority Data
Sep. 29, 2017 (EP) 17194196.6

(51) Int. Cl. C07K 16/22; C07K 16/28; A61P 35/00

(52) U.S. Cl. CPC C07K 16/22; C07K 16/2818; C07K 16/2827; A61K 2039/507; C07K 2317/565; C07K 2317/76; A61P 35/00

(57) ABSTRACT

The disclosure relates to the combined use of certain anti-IGF antibody molecules with PD1 antagonists for the treatment of cancer. It further relates to pharmaceutical compositions and kits comprising such anti-IGF antibody molecules and antagonists.

### FIELD OF THE INVENTION

The present invention relates to a combination therapy in the treatment of cancer and to compounds for use in such a combination therapy. The compounds for combination are an Insulin-like growth factor (IGF) antagonist and a PD-1 antagonist.

### BACKGROUND OF THE INVENTION

There is a very large body of scientific, epidemiological and clinical literature implicating a role for the IGFs in the development, progression and metastasis of many different cancer types (reviewed by Jerome et al., End. Rel. Cancer 10: 561-578, 2003; and Pollak et al., Nature Rev. Can. 4: 505-518, 2004).

Various strategies inhibiting IGF pathways are being employed such as anti-receptor monoclonal antibodies (including ganitumab, cixutumumab, and dalotuzumab), tyrosine kinase inhibitors (TKI, including dual IGF-1 R and InsR tyrosine kinase inhibitors BMS-754807, KW2450, and linsitinib), and anti-IGF ligand antibodies (dusigitumab=MEDI-573, Astra Zeneca/Med Immune). These agents have been tested in the clinic either as monotherapy or in combination with cytotoxic agents and/or other molecularly targeted agents.

Most cancers express IGF-1 receptors, but there is evidence that overexpression of ligands, particularly IGF-2, occurs in neoplasm. IGF-2 has been shown to exert its proliferative/survival signaling in cancer cells through binding to the IGF-1 receptor and the insulin receptor-A (InsR-A). The major advantage of neutralizing antibodies for both IGF-1 and IGF-2 is that the sequestration of the ligands ensures that receptor activation by IGF-1 or IGF-2 does not occur, and eliminates the possibility of activation of the InsR-A by IGF-2. In addition, an IGF-1 and IGF-2 blocking antibody does not interfere with metabolic signaling of insulin through InsR-B. Hence it offers a balanced approach with therapeutic potential in a variety of cancers, avoiding the pitfalls of targeting the IGF-1 R with monoclonal antibodies (mAbs) or TKI.

A large body of evidence suggests that the IGF signaling system is not a bona fide oncogenic driver (such as mutant or altered EGFR, HER2, ALK, BRAF or KRAS), but rather a resistance mechanism activated upon treatment with established therapies. For example, the IGF axis has been implicated as a bypass pathway conferring resistance to EGFR inhibitors, and inhibitors of downstream pathway molecules such as mTOR or MEK. Based on the extensive interactions between the IGF axis and other receptor tyrosine kinase (RTK) signaling networks (including EGFR, HER2, VEGFR, PGDFR, cMET, and ALK), associated with redundancy of pathways driving cellular proliferation and survival, combinations that incorporate IGF-targeted agents with other RTKs and downstream effectors have been investigated. Preclinical evidence further indicates that IGF signaling can protect tumor cells from chemotherapy- or radiotherapy-induced cell death; hence, combinations of IGF axis inhibitors with standard cytotoxic agents have been investigated (Ireland et al., Cancer Res. 76(23): 6851-6863, 2016).

Despite a convincing preclinical rationale, the results of combination trials with IGF-1 R inhibitory drugs and chemotherapy or other targeted therapies have shown limited clinical benefit (Langer et al., J Clin Oncol. 2014; 32(19):2059-66; Fuchs et al., Ann Oncol. 2015; 26(5):921-7, Sclafani et al., J Natl Cancer Inst. 2015; 107(12):djv258; Van Cutsemet al., Clin Cancer Res. 2014; 20(16):4240-50).

Cancer immunotherapy is a branch of oncology in which the immune system is used to treat cancer, which is in stark contrast to existing common methods of treatment in which the tumor is directly excised or treated. This therapeutic concept is based on the identification of a number of proteins on the surface of T-cells which act to inhibit the immune function of these cells. Listed among these proteins is PD-1 (Programmed cell death-1). PD1 is a key regulator of T-cell activity. Recently it has been shown in a range of different cancer settings that antagonistic PD-1 antibodies molecules can be used to stimulate the immune system and thereby treat cancer.

Despite increasing therapeutic approaches, there is still a need for improved treatment options for cancer patients. The efficacy of therapeutic agents can be improved by using combination therapies (in particular in oncology) with other compounds and/or improving the dosage schedule. Even if the concept of combining several therapeutic agents has already been suggested, and although various combination therapies are under investigation and in clinical trials, there is still a need for new and efficient therapeutic concepts for the treatment of cancer diseases, e.g. solid tumors, which show advantages over standard therapies, such as for example better treatment outcome, beneficial effects, superior efficacy and/or improved tolerability, such as e.g. reduced side effects of the combined treatment.

It is therefore an object of the present invention to provide pharmaceutical compositions and methods in cancer therapy for improved therapeutic efficacy and applicability, in particular to provide combination treatments/methods of combination treatment providing certain advantages compared to treatments/methods of treatment currently used and/or known in the prior art. These advantages may include in vivo efficacy (e.g. improved clinical response, extend of the response, increase of the rate of response, duration of response, disease stabilization rate, duration of stabilization, time to disease progression, progression free survival (PFS) and/or overall survival (OS), later occurrence of resistance and the like), safe and well tolerated administration and reduced frequency and severity of adverse events.

### BRIEF SUMMARY OF THE INVENTION

The present invention provides a method for treating a patient with an Insulin-like growth factor (IGF) antagonist, preferably an anti-IGF antibody, and most preferably an anti-IGF-1-IGF-2 antibody together with an antagonist of the Programmed Cell Death 1 (PD-1) signaling pathway (PD-1 being an immuno-inhibitory protein that negatively regulates T cell receptor signals). The treatment is expected to result in a reduction of tumor growth or even to tumor shrinkage. Accordingly, the present invention provides a combination therapy comprising an anti-IGF antibody (e.g., an anti-IGF-1-IGF-2 antibody) and a PD-1 antagonist.

In a detailed aspect, the present invention relates to a method of treating and/or preventing an oncological or hyperproliferative disease, in particular cancer or a tumor disease, comprising administering to a patient in need thereof
a) a therapeutically effective amount of Compound A and
b) a therapeutically effective amount of Compound B,
   wherein
Compound A is an anti-IGF antibody,
   and wherein
Compound B is a PD-1 antagonist.

In a related aspect, the present invention provides Compound A and Compound B, each for use in a method of treating and/or preventing an oncological or hyperproliferative disease, said method comprising administering Compound A and Compound B to a patient in need thereof, wherein
Compound A is an anti-IGF antibody,
   and wherein
Compound B is a PD-1 antagonist.

The present invention further relates to the use of Compound A and Compound B, each for preparing a pharmaceutical composition for treating and/or preventing an oncological or hyperproliferative disease, wherein Compound A and Compound B, are intended for or provided for combined administration of Compound A and Compound B, wherein Compound A and Compound B are defined as above.

In another aspect, the present invention discloses a pharmaceutical composition comprising
a) Compound A and
b) Compound B,
   wherein
Compound A is an anti-IGF antibody,
   and wherein
Compound B is a PD-1 antagonist.

In a further aspect, the present invention relates to a kit comprising

a) a first pharmaceutical composition comprising Compound A and
b) a second pharmaceutical composition comprising Compound B,
wherein Compound A and Compound B are defined as above.

In some embodiments of the invention, compound A is an anti-IGF antibody molecule comprising heavy chain complementary determining regions comprising the amino acid sequences of SEQ ID NO: 40 (HCDR1), SEQ ID NO: 41 (HCDR2), and SEQ ID NO: 42 (HCDR3) and light chain determining regions comprising the amino acid sequences of SEQ ID NO: 43 (LCDR1), SEQ ID NO: 44 (LCDR2), and SEQ ID NO: 45 (LCDR3).

In some embodiments of the invention, the anti-IGF antibody described herein is an anti-IGF antibody molecule comprising a heavy chain variable region comprising the amino acid sequence of SEQ ID NO: 46 and a light chain variable region comprising the amino acid sequence of SEQ ID NO: 47.

In some embodiments of the invention, the anti-IGF antibody described herein is an anti-IGF antibody molecule comprising a heavy chain comprising the amino acid sequence of SEQ ID NO: 48, and a light chain comprising the amino acid sequence of SEQ ID NO: 49 (referred to herein as “BI-IGF”). In some embodiments of the invention, compound B is an anti-PD-1 antibody or an anti-PD-L1 antibody.

In some embodiments of the invention, the anti-PD-1 antibody is selected from the group consisting of pembrolizumab, nivolumab, pidilizumab, PDR-001, BAP049-Clone-B, BAP049-Clone-E, PD1-1, PD1-2, PD1-3, PD1-4, and PD1-5.

In some embodiments of the invention, the anti-PD-L1 antibody is selected from the group consisting of atezolizumab, avelumab and durvalumab.

### DESCRIPTION OF THE FIGURES

FIG. 1: (A) Single agent treatment with Compound B delayed tumor growth, with a median TGI of 71%. Treatment with the combination was more efficacious, resulting in a TGI of 83%. (B) All treatment regimens were well tolerated with no significant body weight loss.

FIG. 2: Amino acid sequences of the variable domains of anti-IGF (BI-IGF) antibody as defined herein. The CDR sequences are underlined: (A) VL BI-IGF (SEQ ID NO: 47); (B) VH BI-IGF (SEQ ID NO:46).

FIG. 3: Amino acid sequences of the variable domains of anti-PD1 antibody molecules (anti-PD1 antibodies PD1-1, PD1-2, PD1-3, PD1-4 and PD1-5 as defined herein). The CDR sequences are underlined. (A) VL PD1-1 (SEQ ID NO: 20); VL PD1-2; (SEQ ID NO: 22); VL PD1-3 (SEQ ID NO: 24); VL PD1-4, (SEQ ID NO: 26); VL PD1-5 (SEQ ID NO: 28); (B) VH PD1-1 (SEQ ID NO: 19); VH PD1-2; (SEQ ID NO: 21); VH PD1-3 (SEQ ID NO: 23); VH PD1-4, (SEQ ID NO: 25); VH PD1-5 (SEQ ID NO: 27).

### DETAILED DESCRIPTION OF THE INVENTION

The present invention relates to a method, compounds for use, use of compounds, pharmaceutical compositions and kits, all referring to the combined therapy or combined provision of Compound A and Compound B, wherein
Compound A is an anti-IGF antibody and wherein
Compound B is a PD-1 antagonist.

The present inventors have surprisingly found that a combination of Compound A and Compound B is able to bring about a therapeutic effect in cancer therapy, such as a reduction, or even shrinkage, of tumor growth as compared to a therapy with Compound A only or Compound B only. Compound A and B work together synergistically and can lead to a reduction of cancer.

Compound A according to the present invention is an anti-IGF antibody, in particular an anti-IGF antibody molecule, which binds preferably to human IGF-1 and/or IGF-2.

Insulin-like growth factor-1 (IGF-1; a 70 amino-acid polypeptide) and insulin-like growth factor-2 (IGF-2; a 67 amino-acid polypeptide) are 7.5 kD soluble factors present in serum that can potently stimulate the growth of many mammalian cells (Pollak et al., Nature Rev. Can. 4: 505-518, 2004). On secretion into the bloodstream the IGFs form complexes with IGF-binding proteins (IGFBPs) which protect them from proteolytic degradation in the serum en route to their target tissues and prevents their association with the IGF receptors. IGFs are also known to be secreted in an autocrine or paracrine manner in target tissues themselves. This is known to occur during normal fetal development where the IGFs play a key role in the growth of tissues, bone and organs. It is also seen in many cancer tissues where there is thought to be paracrine signaling between tumor cells and stromal cells or autocrine IGF production by the tumor cells themselves (LeRoith D et al, Cancer Lett 195(2):127-37, 2003).

IGF-1 and IGF-2 are able to bind to the IGF-1 receptor (IGF-1 R) expressed on many normal tissues, which functionally is a 460 kD heterotetramer consisting of a dimerised alpha- and beta-subunit, with similar affinities (Rubin R et al, Lab Invest 73(3):311-31), 1995). IGF-2 can also bind to the IGF-2 receptor, which is thought to prevent IGF-2 from binding and signaling through the IGF-1 R. In this respect the IGF-2 R has been demonstrated to be a tumor suppressor protein. The IGF-1 R is structurally similar to the insulin receptor which exists in two forms, IR-A and IR B, which differ by an alternatively spliced 12 amino acid exon deletion in the extracellular domain of IR A. IR-B is the predominant IR iso form expressed in most normal adult tissues where it acts to mediate the effects of insulin on metabolism. IR-A on the other hand is known to be highly expressed in developing fetal tissues but not in adult normal tissues. Recent studies have also shown that IR A, but not IR-B, is highly expressed in some cancers. The exon deletion in IR A has no impact on insulin binding but does cause a small conformational change that allows IGF-2 to bind with much higher affinity than for IR-B (Frasca F et al, Mol Cell Biol 19(5):3278-88, 1999; Pandini G et al, J Biol Chem 277(42):39684-95, 2002). Thus, because of its expression in cancer tissues and increased propensity for IGF-2 binding, IR-A may be as important as IGF-1 R in mediating the mitogenic effects of IGF-2 in cancer.

Binding of the IGFs to IGF-1 R triggers a complex intracellular signaling cascade which results in activation of proteins that stimulate proliferation and survival (Pollak et al., Nature Rev. Can. 4: 505-518, 2004). Unlike the EGFR and HER2neu receptors there is no known amplification of the IGF-1 R or IR A receptors in cancers indicating that receptor activation is controlled by the presence of active ligand.

By blocking receptor-ligand binding, ligand-induced receptor signaling through the tyrosine kinase activity of the receptor is reduced or prevented. Antibodies capable of blocking receptor-ligand binding are generally referred to as neutralizing antibodies.

Compound B according to the present invention is an antagonist against a member of the protein Programmed Death 1 (PD-1) family, such as against PD-1 itself or against one of its ligands, PD-L1 or PD-L2. PD-1 is known as an immuno-inhibitory protein that negatively regulates TCR signals (Ishida, Y. et al. (1992) EMBO J. 11:3887-3895; Blank, C. et al. (2006) Immunol. Immunother. 56(6):739-745). The interaction between PD-1 and PD-L1 can act as an immune checkpoint, which can lead to, e.g., a decrease in tumor infiltrating lymphocytes, a decrease in T-cell receptor mediated proliferation, and/or immuno-evasion by cancerous cells (Dong et al. (2003) J. Mol. Med. 81:281-7; Blank et al. (2005) Cancer Immunol. Immunother. 54:307-314; Konishi et al. (2004) Clin. Cancer Res. 10:5094-100). Immune suppression can be reversed by inhibiting the local interaction of PD-1 with PD-L1 or PD-L2; the effect is additive when the interaction of PD-1 with PD-L2 is blocked as well (Iwai et al. (2002) Proc. Nat'l. Acad. Sci USA 99:12293-7; Brown et al. (2003) J. Immunol. 170:1257-66).

PD-1 is an inhibitory member of the extended CD28/CTLA-4 family of T cell regulators. Other members of the CD28 family include CD28, CTLA-4, ICOS and BTLA. PD-1 is suggested to exist as a monomer, lacking the unpaired cysteine residue characteristic of other CD28 family members. PD-1 is expressed on activated B cells, T cells, and monocytes (Okazaki et al. (2002) Curr Opin Immunol 14:391779-82; Bennett et al. (2003) J. Immunol. 170:711-8). Two ligands for PD-1 have been identified, PD-L1 (B7-H1) and PD-L2 (B7-DC), that have been shown to downregulate T cell activation upon binding to PD-1 (Freeman et al. (2000) J. Exp. Med. 192:1027-34; Carter et al. (2002) Eur. J. Immunol. 32:634-43). Both PD-L1 and PD-L2 are B7 homologs that bind to PD-1. PD-L1 is abundant in a variety of human cancers (Dong et al. (2002) Nat. Med. 8:787-9).

The PD-1 gene encodes a 55 kDa type I transmembrane protein that is part of the Ig gene superfamily (Agata et al. (1996) Int Immunol. 8:765-72). The complete PD-1 sequence can be found under GenBank Accession No. U64863. Although structurally similar to CTLA-4, PD-1 lacks the MYPPY motif (SEQ ID NO:39) that is important for B7-1 and B7-2 binding.

In view of the above, monoclonal antibodies as PD-1 antagonists have been developed in recent years for use in therapy, more precisely for treating various diseases, including cancer and infectious diseases (e.g., WO2006/121168; WO2015/112900). Any one of such antibodies can be used according to the present invention.

A PD-1 antagonist within the meaning of this invention is a compound that inhibits the interaction of PD-1 with its receptor(s) or ligand(s).

Preferably, the PD-1 antagonist is an inhibitor of PD-1 or an inhibitor of PD-L1. The PD-1 antagonist may preferably be an anti-PD-1-antibody or an anti-PD-L1-antibody, and more preferably a humanized or fully human anti-PD-1 antibody or a humanized or fully human anti-PD-L1 antibody. Any one of these antibodies may be a recombinant human antibody.

The term “antibody” which may interchangeably be used with “antibody molecule” encompasses various antibody structures, including but not limited to poly- or monoclonal, chimeric, humanized, human, mono-, bi- or multispecific antibodies single chain antibodies, single domain antibodies, and fragmented antibodies (also referred to as antibody fragments), such as Fab, F(ab)₂, F(ab′)₂, Fab′, single chain variable-fragments (scFv) or antigen binding domains of an antibody, so long as they exhibit the desired antigen-binding activity. The term “antibody” shall encompass complete immunoglobulins as they are produced by lymphocytes and for example present in blood sera, monoclonal antibodies secreted by hybridoma cell lines, polypeptides produced by recombinant expression in host cells, which have the binding specificity of immunoglobulins or monoclonal antibodies, and molecules which have been derived from such immunoglobulins, monoclonal antibodies, or polypeptides by further processing while retaining their binding specificity. In particular, the term “antibody” includes complete immunoglobulins comprising two heavy chains and two light chains. The term further encompasses a fragment of an immunoglobulin, like Fab fragments and polypeptides having one or more variable domains derived from an immunoglobulin, like single chain antibodies (scFv), single domain antibodies, and the like.

The antibody may have an effector function, such as ADCC or CDC, that is usually mediated by the Fc part (antibody constant region) of the antibody, or it may have no effector function, e.g. by lacking a Fc part or having a blocked, masked Fc part, in essence a Fc part that is not or insufficiently recognized by immune cells or immune system components, like the complement system.

The antibody or its fragment may be of any type, e.g. IgA, IgD, IgE, IgG, IgM. Preferred is IgG.

The terms “monoclonal antibody” or “monoclonal antibody composition” as used herein refer to a preparation of antibody molecules of a single amino acid composition.

A “recombinant antibody” is an antibody which has been produced by a recombinantly engineered host cell. It is optionally isolated or purified.

A “human antibody” is one which possesses an amino acid sequence which corresponds to that of an antibody produced by a human cell or derived from a non-human source that utilizes human antibody repertoires or other human antibody-encoding sequences. This definition of a human antibody specifically excludes a humanized antibody comprising non-human antigen-binding residues.

The term “recombinant human antibody”, as used herein, is intended to include all human antibodies that are prepared, expressed, created or isolated by recombinant means, such as antibodies isolated from a host cell such as a NS0 or CHO cell or from an animal (e.g. a mouse) that is transgenic for human immunoglobulin genes or antibodies expressed using a recombinant expression vector transfected into a host cell. Such recombinant human antibodies have variable and constant regions in a rearranged form. The recombinant human antibodies according to the invention have been subjected to in vivo somatic hypermutation. Thus, the amino acid sequences of the VH and VL regions of the recombinant antibodies are sequences that, while derived from and related to human germ line VH and VL sequences, may not naturally exist within the human antibody germ line repertoire in vivo.

A “humanized” antibody refers to a chimeric antibody comprising amino acid residues from non-human hypervariable regions (HVRs) and amino acid residues from human FRs. In certain embodiments, a humanized antibody will comprise substantially all of at least one, and typically two, variable domains, in which all or substantially all of the HVRs (e.g. complementary determining regions (CDRs)) correspond to those of a non-human antibody, and all or substantially the entire framework regions (FRs) correspond to those of a human antibody. A humanized antibody optionally may comprise at least a portion of an antibody constant region derived from a human antibody. A “humanized form” of an antibody, e.g. a non-human antibody, refers to an antibody that has undergone humanization.

“Binding” of an a polypeptide (such as an immunoglobulin, an antibody, or generally an antigen-binding molecule or a fragment thereof) means “having affinity for” or “having specificity for” a certain epitope, antigen or protein (or for at least one part, fragment or epitope thereof).

Generally, the term “specificity” refers to the number of different types of antigens or epitopes to which a particular antigen-binding molecule (such as an antibody described herein) can bind. The specificity of an antigen-binding molecule can be determined based on its affinity and/or avidity. The affinity, represented by the equilibrium constant for the dissociation of an antigen with an antigen-binding protein (KD), is a measure for the binding strength between an epitope and an antigen-binding site on the antigen-binding protein: the lesser the value of the KD, the stronger the binding strength between an epitope and the antigen-binding molecule (alternatively, the affinity can also be expressed as the affinity constant (KA), which is 1/I(D). As will be clear to the skilled person, affinity can be determined in a manner known in the art, depending on the specific antigen of interest. Avidity is the measure of the strength of binding between an antigen-binding molecule (such as an immunoglobulin, an antibody, or generally an antigen-binding molecule or a fragment thereof) containing it and the pertinent antigen. Avidity is related to both the affinity between an epitope and its antigen binding site on the antigen-binding molecule and the number of pertinent binding sites present on the antigen-binding molecule.

An epitope is a region of an antigen that is bound by an antigen-binding molecule (such as an antibody described herein). The term “epitope” includes any polypeptide determinant capable of specific binding to an antibody or antigen binding moiety. In certain embodiments, epitope determinants include chemically active surface groupings of molecules such as amino acids, glycan side chains, phosphoryl, or sulfonyl, and, in certain embodiments, may have specific three dimensional structural characteristics, and/or specific charge characteristics. Conformational and non-conformational epitopes are distinguished in that the binding to the former but not the latter is lost in the presence of denaturing solvents. As used herein, the terms “binding” and “specific binding” refer to the binding of the antibody or antigen binding moiety to an epitope of the antigen in an in vitro assay, preferably in a plasmon resonance assay (BIAcore®, GE-Healthcare Uppsala, Sweden) with purified wild-type antigen.

The expressions “variable domain” or “variable region” or Fv as used herein denotes each of the pair of light and heavy chains which is involved directly in binding the antibody to the antigen. The variable domain of a light chain is abbreviated as “VL” and the variable domain of a heavy chain is abbreviated as “VH”. The variable light and heavy chain domains have the same general structure and each domain comprises four framework (FR) regions whose sequences are widely conserved, connected by three HVRs (or CDRs). The framework regions adopt a beta-sheet conformation and the CDRs may form loops connecting the beta-sheet structure. The CDRs in each chain are held in their three-dimensional structure by the framework regions and form together with the CDRs from the other chain the antigen binding site. The antibody's heavy and light chain CDR regions play a particularly important role in the binding specificity/affinity of the antibodies according to the invention and therefore provide a further object of the invention.

Within the context of this invention, reference to CDR's is based on the definition of Chothia (Chothia and Lesk, J. Mol. Biol. 1987, 196: 901-917), together with Kabat (E. A. Kabat, T. T. Wu, H. Bilofsky, M. Reid-Miller and H. Perry, Sequence of Proteins of Immunological Interest, National Institutes of Health, Bethesda (1983)).

The term “constant domains” or “constant region” as used within the current application denotes the sum of the domains of an antibody other than the variable region. The constant region is not directly involved in binding of an antigen, but exhibits various effector functions.

The “constant domains” as used in the antibodies disclosed herein are preferably from human origin, which is from a constant heavy chain region of a human antibody of the subclass IgG1, IgG2, IgG3, or IgG4 and/or a constant light chain kappa or lambda region. Such constant domains and regions are well known in the state of the art and e.g. described by Kabat et al. (“Sequence of proteins of immunological interest”, US Public Health Services, NIH Bethesda, Md., Publication No. 91).

The “Fc part” of an antibody is not involved directly in binding of an antibody to an antigen, but exhibits various effector functions. A “Fc part of an antibody” is a term well known to the skilled artisan and defined on the basis of papain cleavage of antibodies. Depending on the amino acid sequence of the constant region of their heavy chains, antibodies or immunoglobulins are divided in the classes: IgA, IgD, IgE, IgG and IgM, and several of these may be further divided into subclasses (isotypes), e.g. IgG1, IgG2, IgG3, and IgG4, IgA1, and IgA2. According to the heavy chain constant regions the different classes of immunoglobulins are called α,δ,ε,γ and μ, respectively. The Fc part of an antibody is directly involved in ADCC (antibody-dependent cell-mediated cytotoxicity) and CDC (complement-dependent cytotoxicity) based on complement activation, C1q binding and Fc receptor binding. Complement activation (CDC) is initiated by binding of complement factor C1q to the Fc part of most IgG antibody subclasses. While the influence of an antibody on the complement system is dependent on certain conditions, binding to C1q is caused by defined binding sites in the Fc part. Such binding sites are known in the state of the art and described e.g. by Boackle, R. J., et al, Nature 282 (1979) 742-743; Lukas, T. J., et al, J. Immunol. 127 (1981) 2555-2560; Brunhouse, R., and Cebra, J. J., Mol. Immunol. 16 (1979) 907-917; Burton, D. R., et al, Nature 288 (1980) 338-344; Thommesen, J. E., et al, Mol. Immunol. 37 (2000) 995-1004; Idusogie, E. E., et al, J. Immunol. 164 (2000) 4178-4184; Hezareh, M., et al, J. Virology 75 (2001) 12161-12168; Morgan, A., et al, Immunology 86 (1995) 319-324; EP 0 307 434. Such binding sites are e.g. L234, L235, D270, N297, E318, K320, K322, P331 and P329 (numbering according to EU index of Kabat, E. A., see below). Antibodies of subclass IgG1, IgG2 and IgG3 usually show complement activation and C1q and C3 binding, whereas IgG4 do not activate the complement system and do not bind C1q and C3.

When used herein the term “comprising” and variations thereof such as “comprises” and “comprise” can be substituted with the term “containing” or “including” or “having.”

In one aspect, compound A described herein relates to an isolated antibody molecule (e.g. a human or humanized antibody molecule), which a) binds to human IGF-I and IGF-2 such that i) binding of IGF-I and IGF-2 to the IGF-I receptor is prevented and ii) IGF-I receptor-mediated signaling is inhibited, b) binds to mouse and rat IGF-I and IGF-2, c) does not bind to human insulin. Preferably, the anti-IGF antibody molecule within this invention and all its embodiments has heavy chain complementary determining regions comprising the amino acid sequences of SEQ ID NO: 40 (HCDR1), SEQ ID NO: 41 (HCDR2), and SEQ ID NO: 42 (HCDR3) and light chain determining regions comprising the amino acid sequences of SEQ ID NO: 43 (LCDR1), SEQ ID NO: 44 (LCDR2), and SEQ ID NO: 45 (LCDR3).

In another embodiment, the anti-IGF antibody described herein refers to an anti-IGF antibody molecule having a heavy chain variable region comprising the amino acid sequence of SEQ ID NO: 46 and a light chain variable region comprising the amino acid sequence of SEQ ID NO: 47.

In another embodiment, the anti-IGF antibody described herein refers to an anti-IGF antibody molecule having a heavy chain comprising the amino acid sequence of SEQ ID NO: 48, and a light chain comprising the amino acid sequence of SEQ ID NO: 49 (referred to herein as “BI-IGF”).

Preferably, the anti-IGF antibody described herein is a human anti-IGF antibody.

Specifically, the anti-IGF antibody is an antibody molecule defined by the sequences as shown in Table 1 by way of the SEQ ID numbers and respective amino acid sequences, wherein HCDR denotes CDR sequences of the heavy chain variable domain, LCDR denotes CDR sequences of the light chain variable domain, VH denotes the heavy chain variable domain, VL denotes the light chain variable domain, HC denotes the (full length) heavy chain and LC denotes the (full length) light chain:

TABLE 1
SEQ ID NOs of the CDR, VH, VL, HC and LC sequences
of anti-IGF antibodies described herein.

| SEQ | Sequence | |
|---|---|---|
| ID NO | Name | Amino acid sequence |
| 40 | IGF- | SYWMS |
| | HCDR1 | |
| 41 | IGF- | SITSYGSFTYYADSVKG |
| | HCDR2 | |
| 42 | IGF- | NMYTHFDS |
| | HCDR3 | |
| 43 | IGF- | SGSSSNIGSNSVS |
| | LCDR1 | |
| 44 | IGF- | DNSKRPS |
| | LCDR2 | |
| 45 | IGF- | QSRDTYGYYWV |
| | LCDR3 | |
| 46 | VH | QVELVESGGGLVQPGGSLRLSCAASGFTFTSYWMSW |
| | | VRQAPGKGLELVSSITSYGSFTYYADSVKGRFTISR |
| | | DNSKNTLYLQMNSLRAEDTAVYYCARNMYTHFDSWG |
| | | QGTLVTVSS |
| 47 | VL | DIVLTQPPSVSGAPGQRVTISCSGSSSNIGSNSVSW |
| | | YQQLPGTAPKLLIYDNSKRPSGVPDRFSGSKSGTSA |
| | | SLAITGLQSEDEADYYCQSRDTYGYYWVFGGGTKLT |
| | | VLG |
| 48 | HC | QVELVESGGGLVQPGGSLRLSCAASGFTFTSYWMSW |
| | | VRQAPGKGLELVSSITSYGSFTYYADSVKGRFTISR |
| | | DNSKNTLYLQMNSLRAEDTAVYYCARNMYTHFDSWG |
| | | QGTLVTVSSASTKGPSVFPLAPSSKSTSGGTAALGC |
| | | LVKDYFPEPVTVSWNSGALTSGVHTFPAVLQSSGLY |
| | | SLSSVVTVPSSSLGTQTYICNVNHKPSNTKVDKKVE |
| | | PKSCDKTHTCPPCPAPELLGGPSVFLFPPKPKDTLM |
| | | ISRTPEVTCVVVDVSHEDPEVKFNWYVDGVEVHNAK |
| | | TKPREEQYNSTYRVVSVLTVLHQDWLNGKEYKCKVS |
| | | NKALPAPIEKTISKAKGQPREPQVYTLPPSRDELTK |
| | | NQVSLTCLVKGFYPSDIAVEWESNGQPENNYKTTPP |
| | | VLDSDGSFFLYSKLTVDKSRWQQGNVFSCSVMHEAL |
| | | HNHYTQKSLSLSPGK |
| 49 | LC | DIVLTQPPSVSGAPGQRVTISCSGSSSNIGSNSVSW |
| | | YQQLPGTAPKLLIYDNSKRPSGVPDRFSGSKSGTSA |
| | | SLAITGLQSEDEADYYCQSRDTYGYYWVFGGGTKLT |
| | | VLGQPKAAPSVTLFPPSSEELQANKATLVCLISDFY |
| | | PGAVTVAWKGDSSPVKAGVETTTPSKQSNNKYAASS |
| | | YLSLTPEQWKSHRSYSCQVTHEGSTVEKTVAPTECS |

In one aspect the anti-IGF antibody described herein does not bind to human insulin at concentrations that are at least 100-fold higher than the minimum concentration required for binding to human IGF-I or IGF-2.

In another aspect, the property of the anti-IGF antibody molecule described herein is characterized by the fact that the affinity of the anti-IGF antibody molecule to IGF-1 and IGF-2, respectively, is at least 100-fold, and even more than 1000-fold, as compared to its affinity to insulin. Even though at very high doses, e.g. more than 100 mg/kg, weak binding may not be completely excluded, the anti-IGF antibody molecule does not bind to insulin at therapeutic doses.

In some embodiments, the anti-IGF antibody molecules described herein do not affect the mitogenic properties of human insulin that are mediated by its binding to the insulin receptor. (In general, a mitogenic property is defined as the ability of a compound to encourage a cell to commence cell division, triggering mitosis, e.g. in the case of insulin, its ability to promote cell growth).

In some embodiments, in addition to its ability to inhibit IGF signaling mediated via the IGF-1 receptor, the anti-IGF antibody described herein also has the ability to inhibit IGF-2 signaling mediated via the insulin receptor IR-A.

The anti IGF antibodies described herein have surprisingly high neutralisation potency towards IGF-1 and IGF-2. Furthermore, they have an unexpected higher potency and binding affinity towards IGF-1 than towards IGF-2. They have high solubility and stability, they are free of undesirable glycosylation or hydrolysis motifs in the variable domain, and have a long half-life in the circulation.

In some embodiments, the anti-IGF antibodies described herein reduce the immunosuppressive environment in a tumor, for example by reducing the number of regulatory T cells. In the tumor, PD-1 blockade leads to induction of anti-tumor immune responses through activation of CD8+ T cells (cytotoxic T cells, CTL). Without wishing to be bound by any scientific theory, the anti-IGF antibody described herein may further augment the anti-tumor immune response by depletion of regulatory T cells (Tregs) resulting in a reduced immune suppression in the tumor microenvironment.

Manufacture and therapeutic use of the aforementioned anti-IGF antibodies is disclosed in WO 2010/066868, WO 2013/060872 and WO 2014/135611. In particular, these documents provide a sufficient disclosure of the method of preparing the anti-IGF antibody molecule used in the present invention.

In a further aspect, within the present invention it is referred to an anti-IGF antibody molecule as described herein for use as medicament.

In a further aspect, within the present invention it is referred to a pharmaceutical composition comprising, as the active ingredient, an anti-IGF antibody molecule, preferably a full antibody, defined by the CDR, VH, VL, HC and LC sequences shown in Table 1.

The anti-IGF antibody may be administered to the patient at a dose of 1 mg/kg to 50 mg/kg, for example at any one of at least 20 mg/kg, 25 mg/kg, 30 mg/kg, and 35 mg/kg up to any one of 40 mg/kg, 45 mg/kg and 50 mg/kg, by one or more separate administrations, or by continuous infusion, e.g. infusion over 1 hour. A typical treatment schedule usually involves administration of the antibody once every week to once every three weeks.

In some embodiments, the anti-IGF antibody is administered at a dose of any one of at least 20 mg/kg, 25 mg/kg, 30 mg/kg, and 35 mg/kg up to any one of 40 mg/kg, 45 mg/kg and 50 mg/kg weekly, once every two weeks, once every three weeks in a four week treatment cycle. For example, a dose of 15, 20, 25, 30 or 35 mg/kg (e.g., 25 mg/kg) could be administered weekly, every two weeks or once every three weeks. The antibody may be prepared at a concentration of 10 mg/mL to 100 mg/mL of antibody (e.g., at 10 mg/mL, 30, 50, 65, or 75 mg/mL of antibody). The antibody may preferably be administered to a patient as a total dose of at least 750 mg (up to 1000 mg) by one hour i.v. infusion, to be repeated once a week until disease progression.

A PD-1 antagonist within the meaning of this invention and all of its embodiments is a compound that inhibits the interaction of PD-1 with its receptor(s), preferably an anti-PD-1 antibody or an anti-PD-L1 antibody.

Preferably, the PD-1 antagonist, i.e. the anti-PD-1 antibody or anti-PD-L1 antibody within this invention and all its embodiments is a humanized or fully human anti-PD-1 antibody or a humanized or fully human anti-PD-L1 antibody.

PD-1 antagonists are well-known in the art, e.g. reviewed by Li et al., Int. J. Mol. Sci. 2016, 17, 1151 (incorporated herein by reference). Any antagonist, especially antibodies, disclosed by Li et al., as well as the further antibodies disclosed herein below, can be used according to the invention. Preferably, the PD-1 antagonist of this invention and all its embodiments is selected from the group consisting of the following antibodies (B0):
pembrolizumab (anti-PD-1 antibody);
nivolumab (anti-PD-1 antibody);
pidilizumab (anti-PD-1 antibody);
PDR-001 (anti-PD-1 antibody);
PD1-1, PD1-2, PD1-3, PD1-4, and PD1-5 as disclosed herein below and in EP16170174.3 (anti-PD-1 antibodies);
an anti-PD-1 antibody (generically and/or specifically) disclosed in WO 2015/112900:
   any one of the antibodies as defined in table 1 in WO 2015/112900 (page 171)
   any one of the humanized antibodies as defined in table 1 in WO 2015/112900 (page 171)
   any one of BAP049-hum01 to BAP049-hum16 as defined in table 1 in WO 2015/112900 (page 171)
   any one of BAP049-Clone-A to BAP049-Clone-E as defined in table 1 in WO 2015/112900 (page 171);
atezolizumab (anti-PD-L1 antibody);
avelumab (anti-PD-L1 antibody);
durvalumab (anti-PD-L1 antibody);
an anti-PD-L1 antibody (generically and/or specifically) disclosed in WO 2016/061142:
   any one of the antibodies as defined in table 1 in WO 2016/061142 (page 265);
   any one of the humanized antibodies as defined in table 1 in WO 2016/061142 (page 265);
   any one of BAP058-hum01 to BAP058-hum17 as defined in table 1 in WO 2016/061142 (page 265)
   any one of BAP058-Clone-K to BAP058-Clone-O as defined in table 1 in WO 2016/061142 (page 265).

Pembrolizumab (formerly also known as lambrolizumab; trade name Keytruda; also known as MK-3475) disclosed e.g. in Hamid, O. et al. (2013) New England Journal of Medicine 369(2):134-44, is a humanized IgG4 monoclonal antibody that binds to PD-1; it contains a mutation at C228P designed to prevent Fc-mediated cytotoxicity. Pembrolizumab is e.g. disclosed in U.S. Pat. No. 8,354,509 and WO2009/114335. It is approved by the FDA for the treatment of patients suffering from unresectable or metastatic melanoma and patients with metastatic NSCLC.

Nivolumab (CAS Registry Number: 946414-94-4; BMS-936558 or MDX1106b) is a fully human IgG4 monoclonal antibody which specifically blocks PD-1, lacking detectable antibody-dependent cellular toxicity (ADCC). Nivolumab is e.g. disclosed in U.S. Pat. No. 8,008,449 and WO2006/121168. It has been approved by the FDA for the treatment of patients suffering from unresectable or metastatic melanoma, metastatic NSCLC and advanced renal cell carcinoma.

Pidilizumab (CT-011; Cure Tech) is a humanized IgGlk monoclonal antibody that binds to PD-1. Pidilizumab is e.g. disclosed in WO 2009/101611.

PDR-001 or PDR001 is a high-affinity, ligand-blocking, humanized anti-PD-1 IgG4 antibody that blocks the binding of PD-L1 and PD-L2 to PD-1. PDR-001 is disclosed in WO2015/112900 and WO2017/019896.

Antibodies PD1-1 to PD1-5 are antibody molecules defined by the sequences as shown in Table 2 by way of the SEQ ID numbers, wherein VH denotes the heavy chain variable domain, VL denotes the light chain variable domain, HC denotes the (full length) heavy chain and LC denotes the (full length) light chain:

TABLE 2
SEQ ID NOs of the CDR, VH, VL, HC and LC sequences

| anti-PD1 | CDR | VH | VL | HC | LC |
|---|---|---|---|---|---|
| antibody | sequences | sequences | sequences | sequences | sequences |
| PD1-1 | 1-6 | 19 | 20 | 29 | 30 |
| PD1-2 | 7-12 | 21 | 22 | 31 | 32 |
| PD1-3 | 13-18 | 23 | 24 | 33 | 34 |
| PD1-4 | 13-18 | 25 | 26 | 35 | 36 |
| PD1-5 | 13-18 | 27 | 28 | 37 | 38 |

and wherein the amino acid sequences (and sequence names) of the SEQ ID numbers are as shown in Table 3:

TABLE 3

| SEQ | Sequence | |
|---|---|---|
| ID NO: | name | Amino acid sequence |
| 1 | PD1-1HCDR1 | GFTFSASAMS |
| 2 | PD1-1HCDR2 | YISGGGGDTYYSSSVKG |
| 3 | PD1-1HCDR3 | HSNVNYYAMDY |
| 4 | PD1-1LCDR1 | RASENIDTSGISFMN |
| 5 | PD1-1LCDR2 | VASNQGS |
| 6 | PD1-1LCDR3 | QQSKEVPWT |
| 7 | PD1-2HCDR1 | GFTFSASAMS |
| 8 | PD1-2HCDR2 | YISGGGGDTYYSSSVKG |
| 9 | PD1-2HCDR3 | HSNPNYYAMDY |
| 10 | PD1-2LCDR1 | RASENIDTSGISFMN |
| 11 | PD1-2LCDR2 | VASNQGS |
| 12 | PD1-2LCDR3 | QQSKEVPWT |
| 13 | PD1-3HCDR1 | GFTFSKSAMS |
| 14 | PD1-3HCDR2 | YISGGGGDTYYSSSVKG |
| 15 | PD1-3HCDR3 | HSNVNYYAMDY |
| 16 | PD1-3LCDR1 | RASENIDVSGISFMN |
| 17 | PD1-3LCDR2 | VASNQGS |
| 18 | PD1-3LCDR3 | QQSKEVPWT |
| 19 | PD1VH1 | EVMLVESGGGLVQPGGSLRLSCTASGFTFSAS |
| | | AMSWVRQAPGKGLEWVAYISGGGGDTYYSSSV |
| | | KGRFTISRDNAKNSLYLQMNSLRAEDTAVYYC |
| | | ARHSNVNYYAMDYWGQGTLVTVSS |
| 20 | PD1VL1 | EIVLTQSPATLSLSPGERATMSCRASENIDTS |
| | | GISFMNWYQQKPGQAPKLLIYVASNQGSGIPA |
| | | RFSGSGSGTDFTLTISRLEPEDFAVYYCQQSK |
| | | EVPWTFGQGTKLEIK |
| 21 | PD1VH2 | EVMLVESGGGLVQPGGSLRLSCTASGFTFSAS |
| | | AMSWVRQAPGKGLEWVAYISGGGGDTYYSSSV |
| | | KGRFTISRDNAKNSLYLQMNSLRAEDTAVYYC |
| | | ARHSNPNYYAMDYWGQGTLVTVSS |
| 22 | PD1VL2 | EIVLTQSPATLSLSPGERATMSCRASENIDTS |
| | | GISFMNWYQQKPGQAPKLLIYVASNQGSGIPA |
| | | RFSGSGSGTDFTLTISRLEPEDFAVYYCQQSK |
| | | EVPWTFGQGTKLEIK |
| 23 | PD1VH3 | EVMLVESGGGLVQPGGSLRLSCTASGFTFSKS |
| | | AMSWVRQAPGKGLEWVAYISGGGGDTYYSSSV |
| | | KGRFTISRDNAKNSLYLQMNSLRAEDTAVYYC |
| | | ARHSNVNYYAMDYWGQGTLVTVSS |
| 24 | PD1VL3 | EIVLTQSPATLSLSPGERATMSCRASENIDVS |
| | | GISFMNWYQQKPGQAPKLLIYVASNQGSGIPA |
| | | RFSGSGSGTDFTLTISRLEPEDFAVYYCQQSK |
| | | EVPWTFGQGTKLEIK |
| 25 | PD1VH4 | EVMLVESGGGLVQPGGSLRLSCTASGFTFSKS |
| | | AMSWVRQAPGKGLEWVAYISGGGGDTYYSSSV |
| | | KGRFTISRDNAKNSLYLQMNSLRAEDTAVYYC |
| | | ARHSNVNYYAMDYWGQGTLVTVSS |
| 26 | PD1VL4 | EIVLTQSPATLSLSPGERATMSCRASENIDVS |
| | | GISFMNWYQQKPGQAPKLLIYVASNQGSGIPA |
| | | RFSGSGSGTDFTLTISRLEPEDFAVYYCQQSK |
| | | EVPWTFGQGTKLEIK |
| 27 | PD1VH5 | EVMLVESGGGLVQPGGSLRLSCTASGFTFSKS |
| | | AMSWVRQAPGKGLEWVAYISGGGGDTYYSSSV |
| | | KGRFTISRDNAKNSLYLQMNSLRAEDTAVYYC |
| | | ARHSNVNYYAMDYWGQGTLVTVSS |
| 28 | PD1VL5 | EIVLTQSPATLSLSPGERATMSCRASENIDVS |
| | | GISFMNWYQQKPGQAPKLLIYVASNQGSGIPA |
| | | RFSGSGSGTDFTLTISRLEPEDFAVYYCQQSK |
| | | EVPWTFGQGTKLEIK |
| 29 | PD1HC1 | EVMLVESGGGLVQPGGSLRLSCTASGFTFSAS |
| | | AMSWVRQAPGKGLEWVAYISGGGGDTYYSSSV |
| | | KGRFTISRDNAKNSLYLQMNSLRAEDTAVYYC |
| | | ARHSNVNYYAMDYWGQGTLVTVSSASTKGPSV |
| | | FPLAPCSRSTSESTAALGCLVKDYFPEPVTVS |
| | | WNSGALTSGVHTFPAVLQSSGLYSLSSVVTVP |
| | | SSSLGTKTYTCNVDHKPSNTKVDKRVESKYGP |
| | | PCPPCPAPEFLGGPSVFLFPPKPKDTLMISRT |
| | | PEVTCVVVDVSQEDPEVQFNWYVDGVEVHNAK |
| | | TKPREEQFNSTYRVVSVLTVLHQDWLNGKEYK |
| | | CKVSNKGLPSSIEKTISKAKGQPREPQVYTLP |
| | | PSQEEMTKNQVSLTCLVKGFYPSDIAVEWESN |
| | | GQPENNYKTTPPVLDSDGSFFLYSRLTVDKSR |
| | | WQEGNVFSCSVMHEALHNHYTQKSLSLSLG |
| 30 | PD1LC1 | EIVLTQSPATLSLSPGERATMSCRASENIDTS |
| | | GISFMNWYQQKPGQAPKLLIYVASNQGSGIPA |
| | | RFSGSGSGTDFTLTISRLEPEDFAVYYCQQSK |
| | | EVPWTFGQGTKLEIKRTVAAPSVFIFPPSDEQ |
| | | LKSGTASVVCLLNNFYPREAKVQWKVDNALQS |
| | | GNSQESVTEQDSKDSTYSLSSTLTLSKADYEK |
| | | HKVYACEVTHQGLSSPVTKSFNRGEC |
| 31 | PD1HC2 | EVMLVESGGGLVQPGGSLRLSCTASGFTFSAS |
| | | AMSWVRQAPGKGLEWVAYISGGGGDTYYSSSV |
| | | KGRFTISRDNAKNSLYLQMNSLRAEDTAVYYC |
| | | ARHSNPNYYAMDYWGQGTLVTVSSASTKGPSV |
| | | FPLAPCSRSTSESTAALGCLVKDYFPEPVTVS |
| | | WNSGALTSGVHTFPAVLQSSGLYSLSSVVTVP |
| | | SSSLGTKTYTCNVDHKPSNTKVDKRVESKYGP |
| | | PCPPCPAPEFLGGPSVFLFPPKPKDTLMISRT |
| | | PEVTCVVVDVSQEDPEVQFNWYVDGVEVHNAK |
| | | TKPREEQFNSTYRVVSVLTVLHQDWLNGKEYK |
| | | CKVSNKGLPSSIEKTISKAKGQPREPQVYTLP |
| | | PSQEEMTKNQVSLTCLVKGFYPSDIAVEWESN |
| | | GQPENNYKTTPPVLDSDGSFFLYSRLTVDKSR |
| | | WQEGNVFSCSVMHEALHNHYTQKSLSLSLG |
| 32 | PD1LC2 | EIVLTQSPATLSLSPGERATMSCRASENIDTS |
| | | GISFMNWYQQKPGQAPKLLIYVASNQGSGIPA |
| | | RFSGSGSGTDFTLTISRLEPEDFAVYYCQQSK |
| | | EVPWTFGQGTKLEIKRTVAAPSVFIFPPSDEQ |
| | | LKSGTASVVCLLNNFYPREAKVQWKVDNALQS |
| | | GNSQESVTEQDSKDSTYSLSSTLTLSKADYEK |
| | | HKVYACEVTHQGLSSPVTKSFNRGEC |
| 33 | PD1HC3 | EVMLVESGGGLVQPGGSLRLSCTASGFTFSKS |
| | | AMSWVRQAPGKGLEWVAYISGGGGDTYYSSSV |
| | | KGRFTISRDNAKNSLYLQMNSLRAEDTAVYYC |
| | | ARHSNVNYYAMDYWGQGTLVTVSSASTKGPSV |
| | | FPLAPCSRSTSESTAALGCLVKDYFPEPVTVS |
| | | WNSGALTSGVHTFPAVLQSSGLYSLSSVVTVP |
| | | SSSLGTKTYTCNVDHKPSNTKVDKRVESKYGP |
| | | PCPPCPAPEFLGGPSVFLFPPKPKDTLMISRT |
| | | PEVTCVVVDVSQEDPEVQFNWYVDGVEVHNAK |
| | | TKPREEQFNSTYRVVSVLTVLHQDWLNGKEYK |
| | | CKVSNKGLPSSIEKTISKAKGQPREPQVYTLP |
| | | PSQEEMTKNQVSLTCLVKGFYPSDIAVEWESN |
| | | GQPENNYKTTPPVLDSDGSFFLYSRLTVDKSR |
| | | WQEGNVFSCSVMHEALHNHYTQKSLSLSLG |
| 34 | PD1LC3 | EIVLTQSPATLSLSPGERATMSCRASENIDVS |
| | | GISFMNWYQQKPGQAPKLLIYVASNQGSGIPA |
| | | RFSGSGSGTDFTLTISRLEPEDFAVYYCQQSK |
| | | EVPWTFGQGTKLEIKRTVAAPSVFIFPPSDEQ |
| | | LKSGTASVVCLLNNFYPREAKVQWKVDNALQS |
| | | GNSQESVTEQDSKDSTYSLSSTLTLSKADYEK |
| | | HKVYACEVTHQGLSSPVTKSFNRGEC |
| 35 | PD1HC4 | EVMLVESGGGLVQPGGSLRLSCTASGFTFSKS |
| | | AMSWVRQAPGKGLEWVAYISGGGGDTYYSSSV |
| | | KGRFTISRDNAKNSLYLQMNSLRAEDTAVYYC |
| | | ARHSNVNYYAMDYWGQGTLVTVSSASTKGPSV |
| | | FPLAPCSRSTSESTAALGCLVKDYFPEPVTVS |
| | | WNSGALTSGVHTFPAVLQSSGLYSLSSVVTVP |
| | | SSSLGTKTYTCNVDHKPSNTKVDKRVESKYGP |
| | | PCPPCPAPEFLGGPSVFLFPPKPKDTLMISRT |
| | | PEVTCVVVDVSQEDPEVQFNWYVDGVEVHNAK |
| | | TKPREEQFNSTYRVVSVLTVLHQDWLNGKEYK |
| | | CKVSNKGLPSSIEKTISKAKGQPREPQVYTLP |
| | | PSQEEMTKNQVSLTCLVKGFYPSDIAVEWESN |
| | | GQPENNYKTTPPVLDSDGSFFLYSRLTVDKSR |
| | | WQEGNVFSCSVMHEALHNHYTQKSLSLSLG |
| 36 | PD1LC4 | EIVLTQSPATLSLSPGERATMSCRASENIDVS |
| | | GISFMNWYQQKPGQAPKLLIYVASNQGSGIPA |
| | | RFSGSGSGTDFTLTISRLEPEDFAVYYCQQSK |
| | | EVPWTFGQGTKLEIKRTVAAPSVFIFPPSDEQ |
| | | LKSGTASVVCLLNNFYPREAKVQWKVDNALQS |
| | | GNSQESVTEQDSKDSTYSLSSTLTLSKADYEK |
| | | HKVYACEVTHQGLSSPVTKSFNRGEC |
| 37 | PD1HC5 | EVMLVESGGGLVQPGGSLRLSCTASGFTFSKS |
| | | AMSWVRQAPGKGLEWVAYISGGGGDTYYSSSV |
| | | KGRFTISRDNAKNSLYLQMNSLRAEDTAVYYC |
| | | ARHSNVNYYAMDYWGQGTLVTVSSASTKGPSV |
| | | FPLAPCSRSTSESTAALGCLVKDYFPEPVTVS |
| | | WNSGALTSGVHTFPAVLQSSGLYSLSSVVTVP |
| | | SSSLGTKTYTCNVDHKPSNTKVDKRVESKYGP |
| | | PCPPCPAPEFLGGPSVFLFPPKPKDTLMISRT |
| | | PEVTCVVVDVSQEDPEVQFNWYVDGVEVHNAK |
| | | TKPREEQFNSTYRVVSVLTVLHQDWLNGKEYK |
| | | CKVSNKGLPSSIEKTISKAKGQPREPQVYTLP |
| | | PSQEEMTKNQVSLTCLVKGFYPSDIAVEWESN |
| | | GQPENNYKTTPPVLDSDGSFFLYSRLTVDKSR |
| | | WQEGNVFSCSVMHEALHNHYTQKSLSLSLG |
| 38 | PD1LC5 | EIVLTQSPATLSLSPGERATMSCRASENIDVS |
| | | GISFMNWYQQKPGQAPKLLIYVASNQGSGIPA |
| | | RFSGSGSGTDFTLTISRLEPEDFAVYYCQQSK |
| | | EVPWTFGQGTKLEIKRTVAAPSVFIFPPSDEQ |
| | | LKSGTASVVCLLNNFYPREAKVQWKVDNALQS |
| | | GNSQESVTEQDSKDSTYSLSSTLTLSKADYEK |
| | | HKVYACEVTHQGLSSPVTKSFNRGEC |

Specifically, an anti-PD-1 antibody molecule described herein comprises: (a) heavy chain CDRs comprising the amino acid sequence of SEQ ID NO:1 (HCDR1), SEQ ID NO:2 (HCDR2) and SEQ ID NO:3 (HCDR3) and light chain CDRs comprising the amino acid sequence of SEQ ID NO:4 (LCDR1), SEQ ID NO:5 (LCDR2) and SEQ ID NO:6 (LCDR3); or, b) heavy chain CDRs comprising the amino acid sequence of SEQ ID NO:7 (HCDR1), SEQ ID NO:8 (HCDR2) and SEQ ID NO:9 (HCDR3) and light chain CDRs comprising the amino acid sequence of SEQ ID NO:10 (LCDR1), SEQ ID NO:11 (LCDR2) and SEQ ID NO:12 (LCDR3); or (c) heavy chain CDRs comprising the amino acid sequence of SEQ ID NO:13 (HCDR1), SEQ ID NO:14 (HCDR2) and SEQ ID NO:15 (HCDR3) and light chain CDRs comprising the amino acid sequence of SEQ ID NO:16 (LCDR1), SEQ ID NO:17 (LCDR2) and SEQ ID NO:18 (LCDR3).

In some embodiments, the anti-PD-1 antibody molecule has a heavy chain variable domain comprising an amino acid sequence selected from SEQ ID NOs: 19, 21, 23, 25 and 27.

In some embodiments, the anti-PD-1 antibody molecule has a light chain variable domain comprising an amino acid sequence selected from SEQ ID NOs: 20, 22, 24, 26 and 28.

In some embodiments, the anti-PD-1 antibody molecule has (a) a heavy chain variable domain comprising the amino acid sequence of SEQ ID NO: 19 and a light chain variable domain comprising the amino acid sequence of SEQ ID NO: 20, (b) a heavy chain variable domain comprising the amino acid sequence of SEQ ID NO: 21 and a light chain variable domain comprising the amino acid sequence of SEQ ID NO: 22, (c) a heavy chain variable domain comprising the amino acid sequence of SEQ ID NO: 23 and a light chain variable domain comprising the amino acid sequence of SEQ ID NO: 24, (d) a heavy chain variable domain comprising the amino acid sequence of SEQ ID NO: 25 and a light chain variable domain comprising the amino acid sequence of SEQ ID NO: 26, or (e) a heavy chain variable domain comprising the amino acid sequence of SEQ ID NO: 27 and a light chain variable domain comprising the amino acid sequence of SEQ ID NO: 28.

In some embodiments, the anti-PD-1 antibody has (a) a heavy chain comprising the amino acid sequence of SEQ ID NO: 29 and a light chain comprising the amino acid sequence of SEQ ID NO: 30, (b) a heavy chain comprising the amino acid sequence of SEQ ID NO: 31 and a light chain comprising the amino acid sequence of SEQ ID NO: 32, (c) a heavy chain comprising the amino acid sequence of SEQ ID NO: 33 and a light chain comprising the amino acid sequence of SEQ ID NO: 34, (d) a heavy chain comprising the amino acid sequence of SEQ ID NO: 35 and a light chain comprising the amino acid sequence of SEQ ID NO: 36, or (e) a heavy chain comprising the amino acid sequence of SEQ ID NO: 37 and a light chain comprising the amino acid sequence of SEQ ID NO: 38.

Atezolizumab (Tecentriq, also known as MPDL3280A) is a phage-derived human IgGlk monoclonal antibody targeting PD-L1 and is described e.g. in Deng et al. mAbs 2016; 8:593-603. It has been approved by the FDA for the treatment of patients suffering from urothelial carcinoma.

Avelumab is a fully human anti-PD-L1 IgG1 monoclonal antibody and described in e.g. Boyerinas et al. Cancer Immunol. Res. 2015; 3:1148-1157.

Durvalumab (MEDI4736) is a human IgGlk monoclonal antibody with high specificity to PD-L1 and described in e.g. Stewart et al. Cancer Immunol. Res. 2015; 3:1052-1062 or in Ibrahim et al. Semin. Oncol. 2015; 42:474-483.

Further PD-1 antagonists disclosed by Li et al. (supra), or known to be in clinical trials, such as AMP-224, MEDI0680 (AMP-514), REGN2810, BMS-936559, JS001-PD-1, SHR-1210, BMS-936559, TSR-042, JNJ-63723283, MEDI4736, MPDL3280A, and MSB0010718C, may be used as alternative or in addition to the above mentioned antagonists.

The INNs as used herein are meant to also encompass all biosimilar antibodies having the same, or substantially the same, amino acid sequences as the originator antibody, including but not limited to those biosimilar antibodies authorized under 42 USC § 262 subsection (k) in the US and equivalent regulations in other jurisdictions.

PD-1 antagonists listed above are known in the art with their respective manufacture, therapeutic use and properties.

In one embodiment the PD-1 antagonist is pembrolizumab (B1).

In another embodiment the PD-1 antagonist is nivolumab (B2).

In another embodiment the PD-1 antagonist is pidilizumab (B3).

In another embodiment the PD-1 antagonist is atezolizumab (B4).

In another embodiment the PD-1 antagonist is avelumab (B5).

In another embodiment the PD-1 antagonist is durvalumab (B6).

In another embodiment the PD-1 antagonist is PDR-001 (B7).

In another embodiment the PD-1 antagonist is BAP049-Clone-B as defined in table 1 in WO 2015/112900 (page 171) (B8).

In another embodiment the PD-1 antagonist is BAP049-Clone-E as defined in table 1 in WO 2015/112900 (page 171) (B9).

In another embodiment the PD-1 antagonist is selected from the group consisting of BAP058-Clone-K to BAP058-Clone-O as defined in table 1 in WO 2016/061142 (page 265) (B10).

In another embodiment the PD-1 antagonist is PD1-1 (B11) In another embodiment the PD-1 antagonist is PD1-2 (B12).

In another embodiment the PD-1 antagonist is PD1-3 (B13).

In another embodiment the PD-1 antagonist is PD1-4 (B14).

In another embodiment the PD-1 antagonist is PD1-5 (B15).

Administration of Compound B, the PD-1 antagonist, as described herein may e.g. be by injection (e.g. subcutaneously or intravenously) at a dose of about 0.1 to 30 mg/kg of patient body weight, e.g. about 0.5 to 25 mg/kg of patient body weight, about 1 to 20 mg/kg of patient body weight, about 2 to 5 mg/kg of patient body weight, or about 3 mg/kg of patient body weight.

Dosages and therapeutic regimens of the PD-1 antagonist can be determined by a skilled artisan. Preferred dosage regimens for a PD-1 antagonist of the invention include 1 mg/kg of host body weight or 3 mg/kg of host body weight via intravenous administration, with the antibody being given using one of the following dosing schedules: (i) every four weeks for six dosages, then every three months; (ii) every three weeks; (iii) 3 mg/kg of host body weight once followed by 1 mg/kg of host body weight every three weeks. In certain embodiments, the PD-1 antagonist is administered by injection (e.g., subcutaneously or intravenously) at a dose of about 1 to 40 mg/kg of host body weight, e.g., 1 to 30 mg/kg of host body weight, e.g., about 5 to 25 mg/kg of host body weight, about 10 to 20 mg/kg of host body weight, about 1 to 5 mg/kg of host body weight, 1 to 10 mg/kg of host body weight, 5 to 15 mg/kg of host body weight, 10 to 20 mg/kg of host body weight, 15 to 25 mg/kg of host body weight, or about 3 mg/kg of host body weight. The dosing schedule can vary from e.g., once a week to once every 2, 3, or 4 weeks. In one embodiment, the PD-1 antagonist is administered at a dose from about 10 to 20 mg/kg of host body weight every other week. The antibody molecule can be administered by intravenous infusion at a rate of more than 20 mg/min, e.g., 20-40 mg/min, and typically greater than or equal to 40 mg/min to reach a dose of about 35 to 440 mg/m², typically about 70 to 310 mg/m², and more typically, about 110 to 130 mg/m². In embodiments, the infusion rate of about 110 to 130 mg/m² achieves a level of about 3 mg/kg of host body weight. In other embodiments, the antibody molecule can be administered by intravenous infusion at a rate of less than 10 mg/min, e.g., less than or equal to 5 mg/min to reach a dose of about 1 to 100 mg/m², e.g., about 5 to 50 mg/m², about 7 to 25 mg/m², or, about 10 mg/m². In some embodiments, the antibody is infused over a period of about 30 min. It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

To be used in therapy, the antibodies disclosed herein, e.g., the anti-IGF antibody molecule as defined herein, optionally in combination with one or more other active agents (e.g., the PD1 antagonist as described herein) or the anti-PD1 antibody molecule as defined herein, optionally in combination with one or more other active agents (e.g., the anti-IGF antibody as described herein), is included into pharmaceutical compositions appropriate to facilitate administration to animals or humans.

Typical formulations of the anti-IGF and/or PD1 and/or anti-PDL1 antibody molecule can be prepared by mixing the antibody molecule with physiologically acceptable carriers, excipients or stabilizers, in the form of lyophilized or otherwise dried formulations or aqueous solutions or aqueous or non-aqueous suspensions. Carriers, excipients, modifiers or stabilizers are nontoxic at the dosages and concentrations employed. They include buffer systems such as phosphate, citrate, acetate and other inorganic or organic acids and their salts; antioxidants including ascorbic acid and methionine; hydrophilic polymers such as polyvinylpyrrolidone or polyethylene glycol (PEG); stabilizing amino acids such as glycine, glutamine, asparagine, histidine, arginine, or lysine; monosaccharides, disaccharides, oligosaccharides or polysaccharides and other carbohydrates including glucose, mannose, sucrose, trehalose, dextrins or dextrans; chelating agents such as EDTA; sugar alcohols such as, mannitol or sorbitol; salt-forming counter-ions such as sodium; and/or ionic or non-ionic surfactants such as TWEEN™ (polysorbates), PLURONICS™ or fatty acid esters, fatty acid ethers or sugar esters. The excipients may also have a release-modifying or absorption-modifying function.

The antibody molecules may also be dried (freeze-dried, spray-dried, spray-freeze dried, dried by near or supercritical gases, vacuum dried, air-dried).

Naturally, the formulations to be used for in vivo administration must be sterile; sterilization may be accomplished be conventional techniques, e.g. by filtration through sterile filtration membranes.

In a specific embodiment the anti-IGF antibody is formulated in an aqueous buffer composition for parenteral (intravenous) infusion or injection at an antibody concentration of 10 mg/mL, said buffer comprising 24.2 mM histidine, 3.88% mannitole, 0.97% sucrose, 0.02% polysorbate 20, pH 6.0. For intravenous infusion, the pharmaceutical composition may be diluted with a physiological solution, e.g. with 0.9% sodium chloride or G5 solution.

Within this invention it is to be understood that the combinations, compositions, kits, methods, uses or compounds for use according to this invention may envisage the simultaneous, concurrent, sequential, successive, alternate or separate administration of the active agents or components. It will be appreciated that the anti-IGF antibody and the PD-1 antagonist can be administered formulated either dependently or independently, such as e.g. the anti-IGF antibody and the PD-1 antagonist may be administered either as part of the same pharmaceutical composition/dosage form or, preferably, in separate pharmaceutical compositions/dosage forms.

In this context, “combination” or “combined” within the meaning of this invention includes, without being limited, a product that results from the mixing or combining of more than one active agent and includes both fixed and non-fixed (e.g. free) combinations (including kits) and uses, such as e.g. the simultaneous, concurrent, sequential, successive, alternate or separate use of the components or agents. The term “fixed combination” means that the active agents are both administered to a patient simultaneously in the form of a single entity or dosage. The term “non-fixed combination” means that the active agents are both administered to a patient as separate entities either simultaneously, concurrently or sequentially with no specific time limits, wherein such administration provides therapeutically effective levels of the two compounds in the body of the patient. The latter also applies to cocktail therapy, e.g. the administration of three or more active agents.

The administration of the anti-IGF antibody/Compound A and the PD-1 antagonist/Compound B may take place by co-administering the active components or agents, such as e.g. by administering them simultaneously or concurrently in one single or in two separate formulations or dosage forms. Alternatively, the administration of the anti-IGF antibody and the PD-1 antagonist may take place by administering the active components or agents sequentially or in alteration, such as e.g. in two separate formulations or dosage forms.

For example, simultaneous administration includes administration at substantially the same time. This form of administration may also be referred to as “concomitant” administration. Concurrent administration includes administering the active agents within the same general time period, for example on the same day(s) but not necessarily at the same time. Alternate administration includes administration of one agent during a time period, for example over the course of a few days or a week, followed by administration of the other agent during a subsequent period of time, for example over the course of a few days or a week, and then repeating the pattern for one or more cycles. Sequential or successive administration includes administration of one agent during a first time period (for example over the course of a few days or a week) using one or more doses, followed by administration of the other agent during a second time period (for example over the course of a few days or a week) using one or more doses. An overlapping schedule may also be employed, which includes administration of the active agents on different days over the treatment period, not necessarily according to a regular sequence. Variations on these general guidelines may also be employed, e.g. according to the agents used and the condition of the subject.

Accordingly, in a preferred embodiment, in the method according the present invention, Compound A as described herein is administered simultaneously, concurrently, sequentially, successively, alternately or separately with Compound B as described herein. In a similar preferred embodiment, Compound A as described herein for use in a method according to the present invention, is administered simultaneously, concurrently, sequentially, successively, alternately or separately with Compound B as described herein. In a related preferred embodiment, Compound B, as described herein for use in a method according to the present invention, is administered simultaneously, concurrently, sequentially, successively, alternately or separately with Compound A as described herein. In a further preferred embodiment, the use of Compound A as described herein is provided wherein Compound A is to be administered simultaneously, concurrently, sequentially, successively, alternately or separately with Compound B. In a further related preferred embodiment, the use of Compound B as described herein is provided wherein Compound B is to be administered simultaneously, concurrently, sequentially, successively, alternately or separately with Compound A. In another embodiment, the kit according to the present invention is provided wherein the first pharmaceutical composition is to be administered simultaneously, concurrently, sequentially, successively, alternately or separately with the second pharmaceutical composition.

Preferred routes of administration for Compound A, Compound B, or both, administered separately or simultaneously, include, but are not limited to, oral, enterical, parenteral (e.g. intramuscular, intraperitoneal, intravenous, transdermal or subcutaneous injection, or implant), nasal, vaginal, rectal, or topical administration. In a preferred embodiment, the route of administration is intravenous administration, especially intravenous infusion or injection. The compounds of the present invention may be formulated, alone or together, in suitable dosage unit formulations containing conventional non-toxic pharmaceutically acceptable carriers, excipients and/or vehicles appropriate for each route of administration. More preferably, formulations include solid, semi-solid or liquid dosage forms, such as lyophilisation, liquid solutions (e.g. injectable and infusible solutions), dispersions or suspensions, liposomes and suppositories. The preferred mode depends on the intended mode of administration and therapeutic application. Especially preferred embodiments include liquid formulations and lyophilisation. In the case of a lyophilisation, the lyophilisate may be reconstituted in a liquid, preferably water.

Compounds A and B as described herein may be administered daily, 5 times a week, 3 times a week, 2 times a week, once a week, once in 2 weeks, once in 3 weeks, once in 4 weeks. Preferable administration intervals include once a week and once in 2 weeks.

Preferably, Compounds A and B are administered once a week by i.v. infusion.

An administration regimen may include long-term treatment. By “long-term” is meant at least two weeks and preferably, several weeks, months or years of duration. Necessary modifications in this dosage regimen may be determined by one of ordinary skill in the art using only routine experimentation given the teachings herein. See Remington's Pharmaceutical Sciences (Martin, E. W., ed. 4), Mack Publishing Co., Easton, Pa. The dosage can also be adjusted by the individual physician in the event of any complication. Administration may be daily, every second day, every third day, every fourth day, one day per week, two days per week, one day per two weeks, one day per three weeks, etc.

Compounds A and B as described herein may be administered at therapeutically effective amounts in single or divided doses administered at appropriate time intervals. A therapeutically effective amount refers to an amount effective at dosages and for periods of time necessary to achieve the desired therapeutic result and is the minimum amount necessary to prevent, ameliorate, or treat a disease or disorder. A therapeutically effective amount of the compounds described herein may vary according to factors such as the disease state, age, sex, and weight of the individual, and the ability of the compound to elicit a desired response in the individual. A therapeutically effective amount is also one in which any toxic or detrimental effects of the compound is outweighed by the therapeutically beneficial effects. A therapeutically effective dose preferably inhibits a measurable parameter, e.g. a tumor growth rate by at least about 20%, more preferably by at least about 40%, even more preferably by at least about 60%, and still more preferably by at least about 80% relative to untreated subjects or relative to a preceding untreated period of the same subject that is to be treated.

The active compounds may be administered in such doses which are therapeutically effective in monotherapy, or in such doses which are lower or higher than the doses used in monotherapy, but when combined result in a desired (jointly) therapeutically effective amount. The amount of the compounds described herein required for use in treatment may be adapted to the particular compound selected, the route of administration, the nature of the condition being treated and the age and condition of the patient and will be ultimately at the discretion of the attendant physician or clinician. Also, the dosage of the compounds described herein may be adapted depending on the target cell, tumor, tissue, graft, or organ.

The desired dose of Compound A or Compound B may be administered as a fixed amount per administration or as bolus, to reach a set blood concentration in the patient.

The dosing schedule of Compound A and Compound B, separately or together, may vary from e.g. once a week to once every 2, 3 or 4 weeks. In a certain embodiment, the administered amount or dosage of Compound A, Compound B, or both, is lower (e.g. at least 20%, at least 30%, at least 40%, or at least 50% lower). In other embodiments, the amount or dosage of Compound A, Compound B, or both, that results in a desired effect (e.g. treatment of a hyperproliferative or oncological disease) is lower (e.g. at least 20%, at least 30%, at least 40%, or at least 50% lower).

The method, compounds, compounds for use, uses of compounds, pharmaceutical composition and kit as described herein comprises administering to the subject a combination of an anti-IGF antibody molecule and an anti-PD-1 antibody molecule as described herein.

The permutation of embodiments in respect of the anti-IGF antibody with embodiments (B0) to (B15) (in respect of the PD-1 antagonist) results in specific combinations which shall all be deemed to be specifically disclosed and to be embodiments of the invention and of all of its combinations, compositions, kits, methods, uses and compounds for use.

Depending on the cancerous disease to be treated, the combination therapy as defined herein may be used on its own or in further combination with one or more additional therapeutic agents, in particular selected from chemotherapeutic agents or therapeutically active compounds that inhibit angiogenesis, signal transduction pathways or mitotic checkpoints in cancer cells.

The additional therapeutic agent may be administered simultaneously with, optionally as a component of the same pharmaceutical preparation, or before or after administration of the anti-IGF antibody and/or the PD1 antagonist.

The chemotherapeutic agent may be selected from hormones, hormonal analogues and antihormonals (e.g. tamoxifen, toremifene, raloxifene, fulvestrant, megestrol acetate, flutamide, nilutamide, bicalutamide, cyproterone acetate, finasteride, buserelin acetate, fludrocortisone, fluoxymesterone, medroxyprogesterone, octreotide, arzoxifene, pasireotide, vapreotide), aromatase inhibitors (e.g. anastrozole, letrozole, liarozole, exemestane, atamestane, formestane), LHRH agonists and antagonists (e.g. goserelin acetate, leuprolide, abarelix, cetrorelix, deslorelin, histrelin, triptorelin), antimetabolites (e.g. antifolates like methotrexate, pemetrexed, pyrimidine analogues like 5 fluorouracil, capecitabine, decitabine, nelarabine, and gemcitabine, purine and adenosine analogues such as mercaptopurine thioguanine, cladribine and pentostatin, cytarabine, fludarabine); antitumor antibiotics (e.g. anthracyclines like doxorubicin, daunorubicin, epirubicin and idarubicin, mitomycin-C, bleomycin dactinomycin, plicamycin, mitoxantrone, pixantrone, streptozocin); platinum derivatives (e.g. cisplatin, oxaliplatin, carboplatin, lobaplatin, satraplatin); alkylating agents (e.g. estramustine, meclorethamine, melphalan, chlorambucil, busulphan, dacarbazine, cyclophosphamide, Ifosfamide, hydroxyurea, temozolomide, nitrosoureas such as carmustine and lomustine, thiotepa); antimitotic agents (e.g. vinca alkaloids like vinblastine, vindesine, vinorelbine, vinflunine and vincristine; and taxanes like paclitaxel, docetaxel and their formulations, larotaxel; simotaxel, and epothilones like ixabepilone, patupilone, ZK-EPO); topoisomerase inhibitors (e.g. epipodophyllotoxins like etoposide and etopophos, teniposide, amsacrine, topotecan, irinotecan) and miscellaneous chemotherapeutics such as amifostine, anagrelide, interferone alpha, procarbazine, mitotane, and porfimer, bexarotene, celecoxib.

In some embodiments, the treatment involving Compound A and Compound B further includes a “platinum doublet” therapy, i.e. therapy with (i) a platinum compound such as cisplatin or carboplatin, plus (ii) a third-generation chemotherapy agent such as docetaxel, paclitaxel, vinorelbine, or gemcitabine.

In some embodiments, the treatment involving Compound A and Compound B is combined with a cancer cell targeting therapy.

In some embodiments, the combination therapy as described involves Compound A and Compound B without any additional chemotherapeutic agent.

In certain embodiments, the oncological or hyperproliferative disease, in particular cancer or a tumor disease, treated with the combination therapy as disclosed herein, includes but is not limited to, a solid tumor, a hematological cancer (e.g. leukemia, lymphoma, myeloma, e.g. multiple myeloma), and a metastatic lesion. In one embodiment, the cancer is a solid tumor. Examples of solid tumors include malignancies, e.g. sarcomas and carcinomas, e.g. adenocarcinomas of the various organ systems, such as those affecting the lung, breast, ovarian, lymphoid, gastrointestinal (e.g. colon), anal, genitals and genitourinary tract (e.g. renal, urothelial, bladder cells, prostate), pharynx, CNS (e.g. brain, neural or glial cells), head and neck, skin (e.g. melanoma) and pancreas, as well as adenocarcinomas which include malignancies such as colon cancers, rectal cancer, renal-cell carcinoma, liver cancer, non-small cell lung cancer, cancer of the small intestine and cancer of the esophagus. The cancer may be at an early, intermediate, late stage or metastatic cancer.

As used herein, “hyperproliferative disease” refers to conditions wherein cell growth is increased over normal levels. For example, hyperproliferative diseases or disorders include malignant diseases (e.g. esophageal cancer, colon cancer, biliary cancer) and non-malignant diseases (e.g. atherosclerosis, benign hyperplasia, benign prostatic hypertrophy).

In some embodiments, the cancer is chosen from a lung cancer (e.g. NSCLC (e.g. a NSCLC with squamous and/or non-squamous histology, or a NSCLC adenocarcinoma)), a melanoma (e.g. an advanced melanoma), a renal cancer (e.g. a renal cell carcinoma), a liver cancer, a myeloma (e.g. a multiple myeloma), a prostate cancer, a breast cancer (e.g. a breast cancer that does not express one, two or all of estrogen receptor, progesterone receptor, or HER2/neu, e.g. a triple negative breast cancer), a colorectal cancer, a pancreatic cancer, a head and neck cancer (e.g. head and neck squamous cell carcinoma (HNSCC), anal cancer, gastro-esophageal cancer, thyroid cancer, cervical cancer, a lymphoproliferative disease (e.g. a post-transplant lymphoproliferative disease) or a hematological cancer, T-cell lymphoma, B-cell lymphoma, a non-Hodgkin lymphoma, or a leukemia (e.g. a myeloid leukemia or a lymphoid leukemia).

In some embodiments, the cancer is chosen from a carcinoma (e.g. advanced or metastatic carcinoma), melanoma or a lung carcinoma, e.g. a NSCLC.

In some embodiments, the cancer is chosen from a pancreatic cancer, prostate cancer, breast cancer, colorectal cancer, lung cancer, glioblastoma, renal cancer, preferably pancreatic cancer, prostate cancer, breast cancer, colorectal cancer or lung cancer.

In some embodiments, the cancer is pancreatic cancer, lung cancer, breast cancer, melanoma, colorectal cancer, ovarian cancer, gastric cancer, thyroid cancer, liver cancer or prostate cancer.

In some embodiments, the invention relates to methods of treatment of cancer administering compound A (e.g., BI-IGF) and compound B (e.g., any one of B0 to B15), compound A (e.g., BI-IGF) for use in treatment of cancer in combination with compound B (e.g., any one of B0 to B15), compound B (e.g., any one of B0 to B15) for use in treatment of cancer in combination with compound A (e.g., BI-IGF), use of compound A (e.g., BI-IGF) in the manufacture of a medicament for the treatment of cancer in combination with compound B (e.g., any one of B0 to B15), use of compound B (e.g., any one of B0 to B15) in the manufacture of a medicament for the treatment of cancer in combination with compound A (e.g., BI-IGF), a pharmaceutical composition comprising compound A (e.g., BI-IGF) and compound B (e.g., any one of B0 to B15), where the cancer to be treated is a cancer selected from the group consisting of pancreatic cancer, prostate cancer, breast cancer, colorectal cancer, lung cancer, glioblastoma, renal cancer, preferably pancreatic cancer, prostate cancer, breast cancer, colorectal cancer or lung cancer, in particular, NSCLC.

As outlined above, the present invention relates to a pharmaceutical composition comprising Compound A and Compound B as defined herein and to a kit comprising a first pharmaceutical composition comprising Compound A as defined herein and to a second pharmaceutical composition comprising Compound B as defined herein.

The term “pharmaceutical composition” as defined herein refers to a preparation which is in such form as to permit the biological activity of an active ingredient contained therein to be effective, and which contains no additional components which are unacceptably toxic to a subject to which the composition would be administered. A pharmaceutical composition of the present invention can be administered by a variety of methods known in the art. As will be appreciated by the skilled artisan, the route and/or mode of administration will vary depending upon the desired results.

Regardless of the route of administration selected, the Compounds used in a combination therapy of the present invention and/or the pharmaceutical composition, the first pharmaceutical composition and the second pharmaceutical composition of the present invention, are formulated into pharmaceutically acceptable dosage forms by conventional methods known to those of skill in the art.

The kit as defined herein may comprise a suitable container or several suitable containers comprising the first pharmaceutical composition and/or the second pharmaceutical composition, wherein the first and the second pharmaceutical composition may be contained in the same container or in different containers. The kit may be used in any method or any uses of the invention.

Preferably, the kit according to the present invention further comprises a package insert comprising readable instructions for using Compound A and/or Compound B in the treatment and/or prevention of an oncological or hyperproliferative disease, preferably cancer or a tumor disease, in a patient in need thereof. The instructions may provide further detailed as described above with regard to the inventive method and any of its preferred embodiments.

The use of the word “a” or “an” when used in conjunction with the term “comprising” in the claims and/or the specification may mean “one”, but it is also consistent with the meaning of “one or more”, “at least one”, and “one or more than one”.

“About” as used herein means an acceptable degree of error for the quantity measured given the nature of precision of the measurements. Exemplary degrees of error are within 20%, typically within 10%, and more typically within 5% of a given value or range of values.

The term “treating” or “treatment” as used herein means to cure an already present disease state or condition or to increase the likelihood of recovery from the disease state or condition. Treating can also include inhibiting, i.e. arresting the development of a disease state or condition, and ameliorating, i.e. causing regression or delaying progression of a disease. Treatment can be to ameliorate disease symptoms without curing a patient.

The term “preventing” or “prevention” as used herein does not mean to stop a disease state or condition from occurring in a patient or subject completely but may also refer to a reduced risk of developing a disease state or condition.

The present invention is further illustrated by the following examples, without being necessarily limited to these embodiments of the invention. An example or part thereof, including compounds, doses and administration routes, as well as treatment combinations, each as such or in combination with the detailed description above forms part of the invention.

### EXAMPLES

### Example 1

### In Vivo Anti-Tumor Efficacy of the Combination of Compound A and Compound B in a Colon Carcinoma Model

### Material and Methods

The anti-tumor efficacy of the combination of Compound A and Compound B was investigated in a syngeneic mouse tumor model derived from the murine colon carcinoma cell line MC-38. Tumor cells were subcutaneously implanted into immune-competent syngeneic C57Bl/6 six- to eight-week-old female mice (1×10⁶ tumor cells in 30 μl of matrigel), and tumors were established for three days before beginning treatment. Mice were administered intraperitoneally (i.p.) with Compound B (murine antibody to PD-1, 10 mg/Kg, q3or4d), a combination of Compound B (murine antibody to PD-1, 10 mg/Kg, q3or4d) and Compound A (BI-IGF, 200 mg/Kg, q7d), or IgG isotype control antibody (10 mg/Kg, q3or4d), for 15-32 days. Tumor volumes were determined three times a week using a caliper. The volume of each tumor [in mm³] was calculated according to the formula “tumor volume=length*diameter2*π/6”. Percentage of tumor growth inhibition (TGI) values were calculated as follows:

TGI=100×{1−[(treated final day−treated day1)/(control final day−control day1)]}.

The median TGI was determined on day 15. Body weight was measured daily as an indicator of tolerability of treatments.

### Results

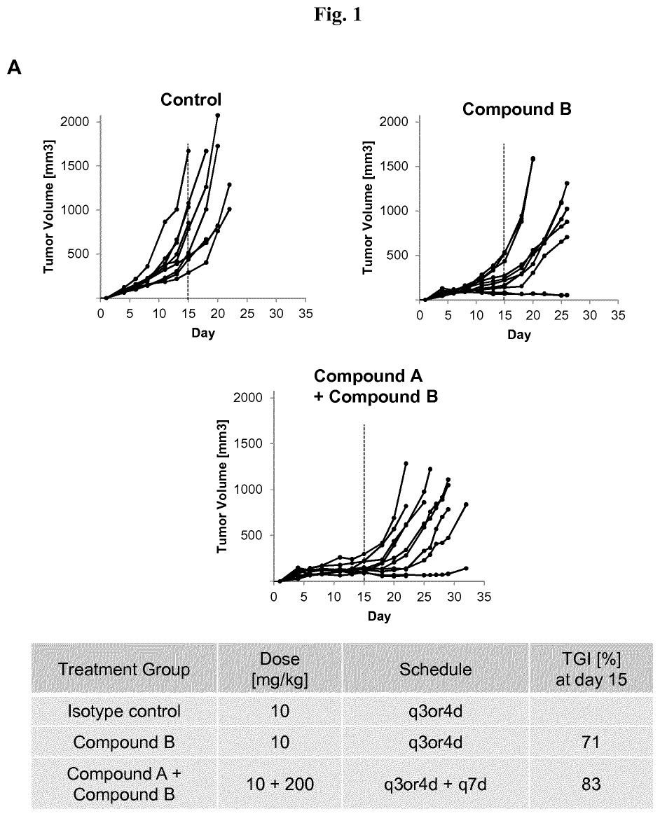

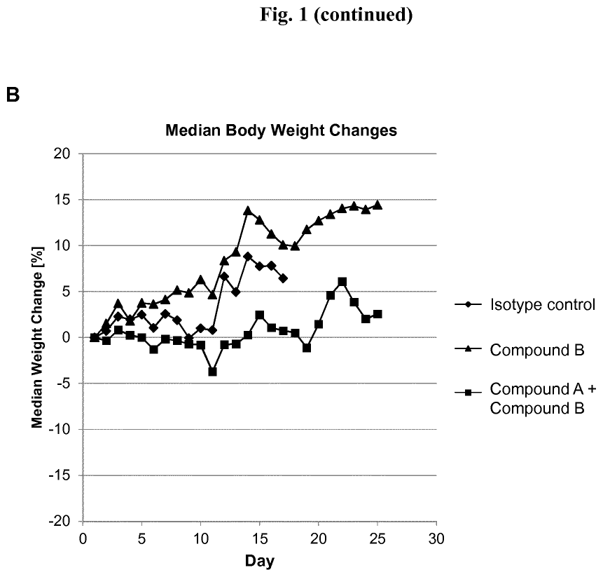

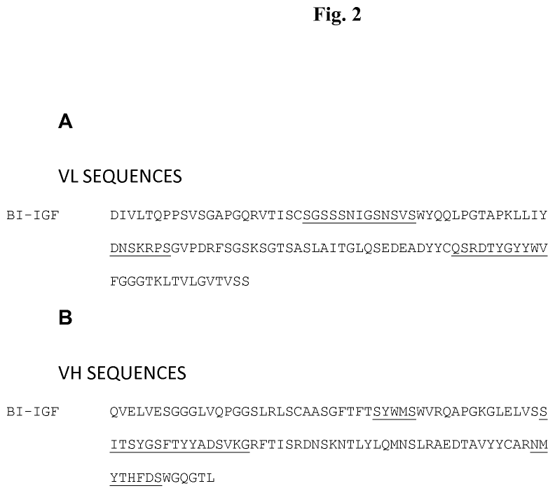

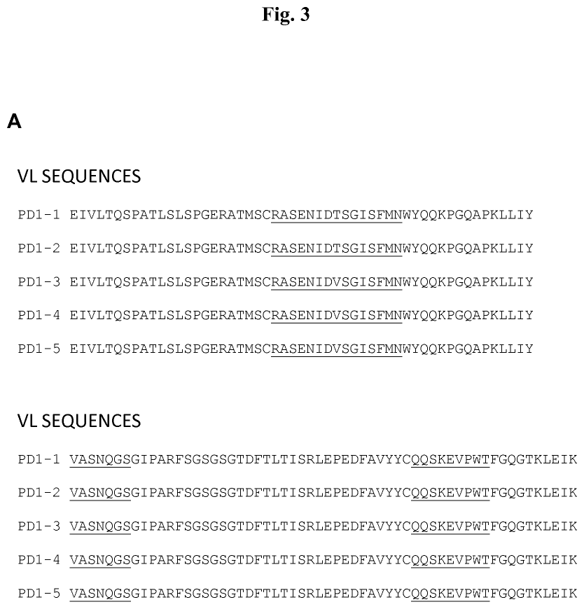

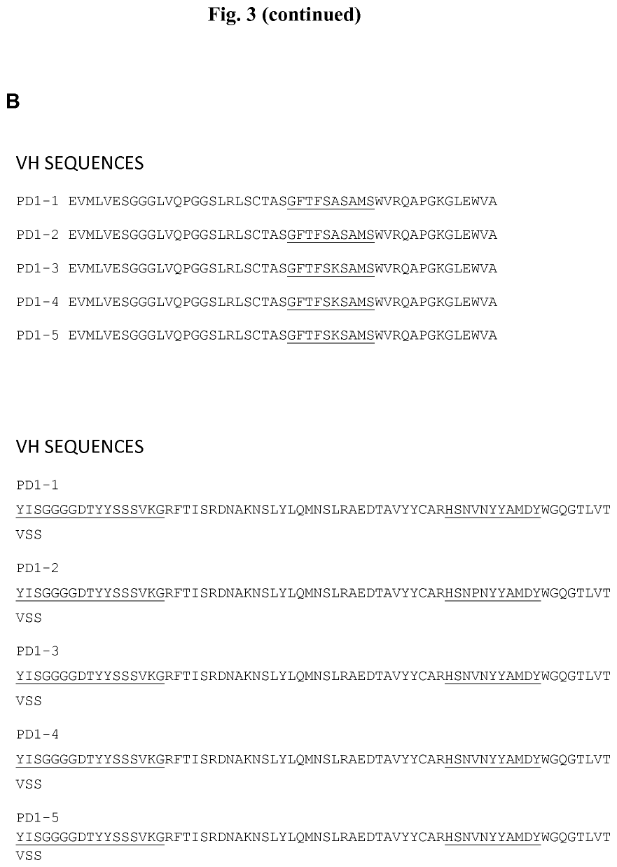

Single agent treatment with Compound B delayed tumor growth, with a median TGI of 71%. Treatment with the combination was more efficacious, resulting in a TGI of 83% (FIG. 1A). All treatment regimens were well tolerated with no significant body weight loss (FIG. 1B).

### Conclusion

The combination of Compound A and Compound B shows superior anti-tumor efficacy when compared to the efficacy of Compound B alone.

### Example 2

### In Vivo Anti-Tumor Efficacy of the Combination of Compound A and Compound B in a Breast Carcinoma Model

### Material and Methods

The anti-tumor efficacy of the combination of Compound A and Compound B is investigated in a syngeneic mouse tumor model derived from the breast mammary carcinoma cell line EMT-6. Tumor cells are subcutaneously implanted into immune-competent syngeneic BALB/c six- to eight-week-old female mice (1×10⁶ tumor cells in 30 μl of matrigel), and tumors are established for six to ten days before beginning treatment. Mice are administered intraperitoneally (i.p.) with Compound B (murine antibody to PD-1, 10 mg/Kg, q3or4d), a combination of Compound B (murine antibody to PD-1, 10 mg/Kg, q3or4d) and Compound A (BI-IGF, 200 mg/Kg, q7d), or IgG isotype control antibody (10 mg/Kg, q3or4d), for 10-35 days. Tumor volumes are determined three times a week using a caliper. The volume of each tumor [in mm³] is calculated according to the formula “tumor volume=length*diameter2*π/6”. Percentage of tumor growth inhibition (TGI) values are calculated as follows:

TGI=100×{1−[(treated final day−treated day1)/(control final day−control day1)]}.

The median TGI is determined on a day after 10 to 15 days of treatment. Body weight is measured daily as an indicator of tolerability of treatments.

### Example 3

### Effect of the Combination of Compound A and Compound B on Intra-Tumoral Regulatory T Cells (Tregs) in a Colon Carcinoma Model

### Material and Methods

The reduction of intra-tumoral Tregs upon treatment with the combination of Compound A and Compound B is investigated in a syngeneic mouse tumor model derived from the colon carcinoma cell line MC-38. Tumor cells are subcutaneously implanted into immune-competent syngeneic C57Bl/6 six- to eight-week-old female mice (1×10⁶ tumor cells in 30 μl of matrigel), and tumors are established for three to six days before beginning treatment. Mice are administered intraperitoneally (i.p.) with Compound A (BI-IGF, 200 mg/Kg, q7d), Compound B (murine antibody to PD-1, 10 mg/Kg, q3or4d), a combination of Compound B (murine antibody to PD-1, 10 mg/Kg, q3or4d) and Compound A (BI-IGF, 200 mg/Kg, q7d), or IgG isotype control antibody (10 mg/Kg, q3or4d), for 10-35 days. Tumor volumes are determined three times a week using a caliper. The volume of each tumor [in mm³] is calculated according to the formula “tumor volume=length*diameter2*n/6”. On the final day of the experiment, when tumor volumes have reached approximately 500-600 mm3, tumors are harvested. One half of the tumors are snap-frozen, the other half is formalin-fixed and paraffin-embedded (FFPE). For assessment of intra-tumoral Tregs, frozen samples are lysed and analyzed by flow cytometry (FACS), and FFPE samples are sectioned and analyzed by immunohistochemistry (IHC).

1. A method of treating or preventing an oncological or hyperproliferative disease comprising administering to a patient in need thereof:
a) a therapeutically effective amount of a Compound A and
b) a therapeutically effective amount of a Compound B,
wherein Compound A is an anti-IGF antibody, and
wherein Compound B is a PD-1 antagonist.

2. The method according to claim 1, wherein the oncological or hyperproliferative disease is cancer or a tumor disease.

3. The method according to claim 1, wherein the oncological or hyperproliferative disease is pancreatic cancer, prostate cancer, breast cancer, colorectal cancer or lung cancer.

4. The method according to claim 1, wherein Compound A is an antibody molecule, which binds to human IGF-I and IGF-2.

5. The method according to claim 1, wherein Compound A is an antibody molecule comprising heavy chain complementary determining regions of SEQ ID NO: 40 (HCDR1), SEQ ID NO: 41 (HCDR2), and SEQ ID NO: 42 (HCDR3) and light chain determining regions of SEQ ID NO: 43 (LCDR1), SEQ ID NO: 44 (LCDR2), and SEQ ID NO: 45 (LCDR3), or wherein Compound A is an antibody molecule comprising a heavy chain variable region of SEQ ID NO: 46 and a light chain variable region of SEQ ID NO: 47, or wherein Compound A is an antibody molecule comprising a heavy chain of SEQ ID NO: 48, and a light chain of SEQ ID NO: 49.

6. The method according to claim 1, wherein the PD-1 antagonist is an anti-PD-1 antibody or an anti-PD-L1 antibody.

7. The method according to claim 6, wherein the anti-PD-1 antibody is selected from the group consisting of pembrolizumab, nivolumab, pidilizumab, PDR-001, BAP049-Clone-B, BAP049-Clone-E, PD1-1, PD1-2, PD1-3, PD1-4, and PD1-5.

8. The method according to claim 6, wherein the anti-PD-L1 antibody is selected from the group consisting of atezolizumab, avelumab and durvalumab.

9. The method according to claim 1, wherein Compound A is administered simultaneously, concurrently, sequentially, successively, alternately or separately with Compound B.

10-17. (canceled)

18. A pharmaceutical composition comprising:
a) Compound A and
b) Compound B,
wherein Compound A is an anti-IGF antibody,
and wherein Compound B is a PD-1 antagonist.

19. The pharmaceutical composition according to claim 18, wherein Compound A is an antibody molecule, which binds to human IGF-I and IGF-2.

20. The pharmaceutical composition according to claim 18, wherein Compound A is an antibody molecule comprising heavy chain complementary determining regions of SEQ ID NO: 40 (HCDR1), SEQ ID NO: 41 (HCDR2), and SEQ ID NO: 42 (HCDR3) and light chain determining regions of SEQ ID NO: 43 (LCDR1), SEQ ID NO: 44 (LCDR2), and SEQ ID NO: 45 (LCDR3), or wherein Compound A is a human antibody molecule comprising a heavy chain variable region of SEQ ID NO: 46 and a light chain variable region of SEQ ID NO: 47, or wherein Compound A is a human antibody molecule comprising a heavy chain of SEQ ID NO: 48, and a light chain of SEQ ID NO: 49.

21. The pharmaceutical composition according to claim 18, wherein the PD-1 antagonist is an anti-PD-1 antibody or an anti-PD-L1 antibody.

22. The pharmaceutical composition according to claim 21, wherein the anti-PD-1 antibody is selected from the group consisting of pembrolizumab, nivolumab, pidilizumab, PDR-001, BAP049-Clone-B, BAP049-Clone-E, PD1-1, PD1-2, PD1-3, PD1-4, and PD1-5.

23. The pharmaceutical composition according to claim 22, wherein the anti-PD-L1 antibody is selected from the group consisting of atezolizumab, avelumab and durvalumab.

24. The pharmaceutical composition according to claim 18, further comprising one or more pharmaceutically acceptable carriers, excipients and/or vehicles.

25. (canceled)

26. A kit comprising:
a) a first pharmaceutical composition or dosage form comprising Compound A and
b) a second pharmaceutical composition or dosage form comprising Compound B,
wherein Compound A is an anti-IGF antibody,
and wherein Compound B is a PD-1 antagonist.

27. The kit according to claim 26, wherein Compound A is an antibody molecule, which binds to human IGF-I and IGF-2.

28. The kit according to claim 26, wherein Compound A is an antibody molecule comprising heavy chain complementary determining regions of SEQ ID NO: 40 (HCDR1), SEQ ID NO: 41 (HCDR2), and SEQ ID NO: 42 (HCDR3) and light chain determining regions of SEQ ID NO: 43 (LCDR1), SEQ ID NO: 44 (LCDR2), and SEQ ID NO: 45 (LCDR3), or wherein Compound A is an antibody molecule comprising a heavy chain variable region of SEQ ID NO: 46 and a light chain variable region of SEQ ID NO: 47, or wherein Compound A is an antibody molecule comprising a heavy chain of SEQ ID NO: 48, and a light chain of SEQ ID NO: 49.

29. The kit according to claim 26, wherein the PD-1 antagonist is an anti-PD-1 antibody or an anti-PD-L1 antibody.

30. The kit according to claim 29, wherein the anti-PD-1 antibody is selected from the group consisting of pembrolizumab, nivolumab, pidilizumab, PDR-001, BAP049-Clone-B, BAP049-Clone-E, PD1-1, PD1-2, PD1-3, PD1-4, and PD1-5.

31. The kit according to claim 29, wherein the anti-PD-L1 antibody is selected from the group consisting of atezolizumab, avelumab and durvalumab.

32. The kit according to claim 26, further comprising a package insert comprising readable instructions for simultaneous, concurrent, sequential, successive, alternate or separate administration to a patient in the treatment or prevention of an oncological or hyperproliferative disease, preferably or cancer or a tumor disease in a patient in need thereof.

33. The method according to claim 1, wherein the oncological disease to be treated is a cancer selected from the group consisting of pancreatic cancer, prostate cancer, breast cancer, colorectal cancer, lung cancer, glioblastoma, renal cancer, preferably pancreatic cancer, prostate cancer, breast cancer, colorectal cancer, lung cancer, or NSCLC.

34. The kit according to claim 32, wherein the oncological disease to be treated is a cancer selected from the group consisting of pancreatic cancer, prostate cancer, breast cancer, colorectal cancer, lung cancer, glioblastoma, renal cancer, preferably pancreatic cancer, prostate cancer, breast cancer, colorectal cancer, lung cancer or NSCLC.


SEQUENCE LISTING (49 sequences)

SEQ ID NO: 1 - protein, 10 aa, Artificial Sequence
  CDR sequence
GFTFSASAMS

SEQ ID NO: 2 - protein, 17 aa, Artificial Sequence
  CDR sequence
YISGGGGDTYYSSSVKG

SEQ ID NO: 3 - protein, 11 aa, Artificial Sequence
  CDR sequence
HSNVNYYAMDY

SEQ ID NO: 4 - protein, 15 aa, Artificial Sequence
  CDR sequence
RASENIDTSGISFMN

SEQ ID NO: 5 - protein, 7 aa, Artificial Sequence
  CDR sequence
VASNQGS

SEQ ID NO: 6 - protein, 9 aa, Artificial Sequence
  CDR sequence
QQSKEVPWT

SEQ ID NO: 7 - protein, 10 aa, Artificial Sequence
  CDR sequence
GFTFSASAMS

SEQ ID NO: 8 - protein, 17 aa, Artificial Sequence
  CDR sequence
YISGGGGDTYYSSSVKG

SEQ ID NO: 9 - protein, 11 aa, Artificial Sequence
  CDR sequence
HSNPNYYAMDY

SEQ ID NO: 10 - protein, 15 aa, Artificial Sequence
  CDR sequence
RASENIDTSGISFMN

SEQ ID NO: 11 - protein, 7 aa, Artificial Sequence
  CDR sequence
VASNQGS

SEQ ID NO: 12 - protein, 9 aa, Artificial Sequence
  CDR sequence
QQSKEVPWT

SEQ ID NO: 13 - protein, 10 aa, Artificial Sequence
  CDR sequence
GFTFSKSAMS

SEQ ID NO: 14 - protein, 17 aa, Artificial Sequence
  CDR sequence
YISGGGGDTYYSSSVKG

SEQ ID NO: 15 - protein, 11 aa, Artificial Sequence
  CDR sequence
HSNVNYYAMDY

SEQ ID NO: 16 - protein, 15 aa, Artificial Sequence
  CDR sequence
RASENIDVSGISFMN

SEQ ID NO: 17 - protein, 7 aa, Artificial Sequence
  CDR sequence
VASNQGS

SEQ ID NO: 18 - protein, 9 aa, Artificial Sequence
  CDR sequence
QQSKEVPWT

SEQ ID NO: 19 - protein, 120 aa, Artificial Sequence
  Immunoglobulin domain
EVMLVESGGGLVQPGGSLRLSCTASGFTFSASAMSWVRQAPGKGLEWVAYISGGGGDTYYSSSVKGRFTISRDNAKNSLYLQMNSLRAEDTAVYYCARHSNVNYYAMDYWGQGTLVTVSS

SEQ ID NO: 20 - protein, 111 aa, Artificial Sequence
  Immunoglobulin domain
EIVLTQSPATLSLSPGERATMSCRASENIDTSGISFMNWYQQKPGQAPKLLIYVASNQGSGIPARFSGSGSGTDFTLTISRLEPEDFAVYYCQQSKEVPWTFGQGTKLEIK

SEQ ID NO: 21 - protein, 120 aa, Artificial Sequence
  Immunoglobulin domain
EVMLVESGGGLVQPGGSLRLSCTASGFTFSASAMSWVRQAPGKGLEWVAYISGGGGDTYYSSSVKGRFTISRDNAKNSLYLQMNSLRAEDTAVYYCARHSNPNYYAMDYWGQGTLVTVSS

SEQ ID NO: 22 - protein, 111 aa, Artificial Sequence; identical to SEQ ID NO: 20
  Immunoglobulin domain

SEQ ID NO: 23 - protein, 120 aa, Artificial Sequence
  Immunoglobulin domain
EVMLVESGGGLVQPGGSLRLSCTASGFTFSKSAMSWVRQAPGKGLEWVAYISGGGGDTYYSSSVKGRFTISRDNAKNSLYLQMNSLRAEDTAVYYCARHSNVNYYAMDYWGQGTLVTVSS

SEQ ID NO: 24 - protein, 111 aa, Artificial Sequence
  Immunoglobulin domain
EIVLTQSPATLSLSPGERATMSCRASENIDVSGISFMNWYQQKPGQAPKLLIYVASNQGSGIPARFSGSGSGTDFTLTISRLEPEDFAVYYCQQSKEVPWTFGQGTKLEIK

SEQ ID NO: 25 - protein, 120 aa, Artificial Sequence; identical to SEQ ID NO: 23
  Immunoglobulin domain

SEQ ID NO: 26 - protein, 111 aa, Artificial Sequence; identical to SEQ ID NO: 24
  Immunoglobulin domain

SEQ ID NO: 27 - protein, 120 aa, Artificial Sequence; identical to SEQ ID NO: 23
  Immunoglobulin domain

SEQ ID NO: 28 - protein, 111 aa, Artificial Sequence; identical to SEQ ID NO: 24
  Immunoglobulin domain

SEQ ID NO: 29 - protein, 446 aa, Artificial Sequence
  Antibody chain
EVMLVESGGGLVQPGGSLRLSCTASGFTFSASAMSWVRQAPGKGLEWVAYISGGGGDTYYSSSVKGRFTISRDNAKNSLYLQMNSLRAEDTAVYYCARHSNVNYYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 30 - protein, 218 aa, Artificial Sequence
  Antibody chain
EIVLTQSPATLSLSPGERATMSCRASENIDTSGISFMNWYQQKPGQAPKLLIYVASNQGSGIPARFSGSGSGTDFTLTISRLEPEDFAVYYCQQSKEVPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 31 - protein, 446 aa, Artificial Sequence
  Antibody chain
EVMLVESGGGLVQPGGSLRLSCTASGFTFSASAMSWVRQAPGKGLEWVAYISGGGGDTYYSSSVKGRFTISRDNAKNSLYLQMNSLRAEDTAVYYCARHSNPNYYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 32 - protein, 218 aa, Artificial Sequence; identical to SEQ ID NO: 30
  Antibody chain

SEQ ID NO: 33 - protein, 446 aa, Artificial Sequence
  Antibody chain
EVMLVESGGGLVQPGGSLRLSCTASGFTFSKSAMSWVRQAPGKGLEWVAYISGGGGDTYYSSSVKGRFTISRDNAKNSLYLQMNSLRAEDTAVYYCARHSNVNYYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 34 - protein, 218 aa, Artificial Sequence
  Antibody chain
EIVLTQSPATLSLSPGERATMSCRASENIDVSGISFMNWYQQKPGQAPKLLIYVASNQGSGIPARFSGSGSGTDFTLTISRLEPEDFAVYYCQQSKEVPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 35 - protein, 446 aa, Artificial Sequence; identical to SEQ ID NO: 33
  Antibody chain

SEQ ID NO: 36 - protein, 218 aa, Artificial Sequence; identical to SEQ ID NO: 34
  Antibody chain

SEQ ID NO: 37 - protein, 446 aa, Artificial Sequence; identical to SEQ ID NO: 33
  Antibody chain

SEQ ID NO: 38 - protein, 218 aa, Artificial Sequence; identical to SEQ ID NO: 34
  Antibody chain

SEQ ID NO: 39 - protein, 5 aa, Artificial Sequence
  Binding Motif
MYPPY

SEQ ID NO: 40 - protein, 5 aa, Artificial Sequence
  CDR sequence
SYWMS

SEQ ID NO: 41 - protein, 17 aa, Artificial Sequence
  CDR sequence
SITSYGSFTYYADSVKG

SEQ ID NO: 42 - protein, 8 aa, Artificial Sequence
  CDR sequence
NMYTHFDS

SEQ ID NO: 43 - protein, 13 aa, Artificial Sequence
  CDR sequence
SGSSSNIGSNSVS

SEQ ID NO: 44 - protein, 7 aa, Artificial Sequence
  CDR sequence
DNSKRPS

SEQ ID NO: 45 - protein, 11 aa, Artificial Sequence
  CDR sequence
QSRDTYGYYWV

SEQ ID NO: 46 - protein, 117 aa, Artificial Sequence
  Immunoglobulin domain
QVELVESGGGLVQPGGSLRLSCAASGFTFTSYWMSWVRQAPGKGLELVSSITSYGSFTYYADSVKGRFTISRDNSKNTLYLQMNSLRAEDTAVYYCARNMYTHFDSWGQGTLVTVSS

SEQ ID NO: 47 - protein, 111 aa, Artificial Sequence
  Immunoglobulin domain
DIVLTQPPSVSGAPGQRVTISCSGSSSNIGSNSVSWYQQLPGTAPKLLIYDNSKRPSGVPDRFSGSKSGTSASLAITGLQSEDEADYYCQSRDTYGYYWVFGGGTKLTVLG

SEQ ID NO: 48 - protein, 447 aa, Artificial Sequence
  Antibody chain
QVELVESGGGLVQPGGSLRLSCAASGFTFTSYWMSWVRQAPGKGLELVSSITSYGSFTYYADSVKGRFTISRDNSKNTLYLQMNSLRAEDTAVYYCARNMYTHFDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 49 - protein, 216 aa, Artificial Sequence
  Antibody chain
DIVLTQPPSVSGAPGQRVTISCSGSSSNIGSNSVSWYQQLPGTAPKLLIYDNSKRPSGVPDRFSGSKSGTSASLAITGLQSEDEADYYCQSRDTYGYYWVFGGGTKLTVLGQPKAAPSVTLFPPSSEELQANKATLVCLISDFYPGAVTVAWKGDSSPVKAGVETTTPSKQSNNKYAASSYLSLTPEQWKSHRSYSCQVTHEGSTVEKTVAPTECS